(19) United States
(12) Patent Application Publication
(10) Pub. No.: US 2014/0212949 A1
(43) Pub. Date: Jul. 31, 2014

(54) METHOD FOR PREPARING MICROVESICULAR ADAM15

(71) Applicant: INDUSTRY-ACADEMIC COOPERATION FOUNDATION, YONSEI UNIVERSITY, Seoul (KR)

(72) Inventors: Doo Sik Kim, Seoul (KR); Hee Doo Lee, Seoul (KR); Yeon Hyang Kim, Seoul (KR); Bon Hun Koo, Seoul (KR); Ok Hee Jeon, Seoul (KR)

(73) Assignee: INDUSTRY ACADEMIC COOPERATION FOUNDATION YONSEI UNIVERSITY, Seoul (KR)

(21) Appl. No.: 14/125,622

(22) PCT Filed: Sep. 20, 2012
(86) PCT No.: PCT/KR2012/007549

§ 371 Date: Dec. 12, 2013

(30) Foreign Application Priority Data
Sep. 20, 2011 (KR) 10-2011-0094376

(51) Int. Cl. C12N 9/64

(52) U.S. Cl. CPC C12N 9/6489
USPC 435/226

(57) ABSTRACT

The present invention relates to a method for preparing microvesicular ADAM15, comprising: (a) a step of activating protein kinase C (PKC) of separated mammalian cells; and (b) a step of separating microvesicle-containing ADAM15 from the mammalian cells. According to the present invention, a method for preparing microvesicular ADAM15 from mammalian cells is provided, and a novel cell regulation mechanism mediated by ADAM proteins is provided by the study of the function of the microvesicular ADAM15. Further, the present invention may provide a novel anti-cancer drug using microvesicular ADAM15, which inhibits adhesion, proliferation and migration of tumor cells.

### TECHNICAL FIELD

The present disclosure relates to a method for preparing microvesicular ADAM15 (A Disintegrin And Metalloprotease 15).

### BACKGROUND ART

ADAM (A Disintegrin And Metalloprotease) proteins are membrane-anchored glycoproteins composed of propeptide, metalloprotease, disintegrin-like, cysteine-rich, epidermal growth factor-like, transmembrane, and cytoplasmic domains (Mochizuki and Okada, 2007; Wolfsberg et al., 1995). Human ADAM15, the only ADAM family member to contain an Arg-Gly-Asp (RGD) motif in its disintegrin-like domain (Herren et al., 1997; Kratzschmar et al., 1996), is widely expressed and involved in both tumor progression and suppression. ADAM15 is highly expressed in several tumors and promotes tumor growth and metastasis through the ectodomain shedding of cadherin, TGF-α, and amphiregulin (Najy et al., 2008a; Najy et al., 2008b; Schafer et al., 2004a; Schafer et al., 2004b). In contrast, several studies have shown that ADAM15 overexpression reduces tumor progression (Chen et al., 2008; Toquet et al., 2010; Ungerer et al., 2010). It has also been demonstrated that recombinant disintegrin-like domain of ADAM15 binds to integrins αvβ3 and α5β1 (Nath et al., 1999; Zhang et al., 1998), suppressing both tumor growth and metastasis (Lu et al., 2007; Trochon-Joseph et al., 2004; Wu et al., 2009a; Wu et al., 2008; Zibert et al., 2011). However, the functional mechanism of ADAM15 is not clearly understood yet.

Plasma membrane proteins can be secreted into the extracellular space through ectodomain shedding and microvesicle release (van Kilsdonk et al., 2010). Ectodomain shedding involves a proteolytic cleavage event on the cell surface. Numerous growth factors, receptors, and adhesion molecules are released from the plasma membrane by this process with the cleaved forms possessing various biological functions (Arribas and Borroto, 2002). Microvesicles are also released from the cell membrane and consist of shedding vesicles and exosomes (Cocucci et al., 2009; Thery et al., 2009). Unlike shedding vesicles, which are released by budding at the plasma membrane, exosomes are generated in multivesicular bodies through endocytic pathways and are then released when these bodies fuse with the plasma membrane. Exosomes have conserved sizes (40-60 nm) and densities (1.11-1.18 g/ml), whereas shedding vesicles are heterogeneous in size (up to 1 μm) (Cocucci et al., 2009; Thery et al., 2009). The importance of microvesicular functions in communication, regulation, and changes in cellular genetic information has become of increasing importance (Cocucci et al., 2009; Hawari et al., 2004; Thery et al., 2009; Valadi et al., 2007).

In this study, we demonstrate that ADAM15 is released into the extracellular space as a component of the exosome, and the release of exosomal ADAM15 is enhanced by phorbol 12-myristate 13-acetate (PMA), a typical protein kinase C (PKC) activator (Choi et al., 2006), in various tumor cell types. Moreover, human monocyte-derived macrophages were shown to release significant level of exosomal ADAM15 without PMA stimulation. Functional investigation of exosomal ADAM15 reveals a novel mechanism of regulating cellular processes mediated by the ADAM protein.

Throughout the specification, a number of publications and patent documents are referred to and cited. The disclosure of the cited publications and patent documents is incorporated herein by reference in its entirety to more clearly describe the state of the related art and the present disclosure.

### DISCLOSURE

### Technical Problem

The present inventors investigated a biological importance of ADAM 15 (a disintegrin and metalloprotease 15), as an exosomal component, in the tumor suppression mechanism associated with the immune function of ADAM15, and then endeavored to develop a method for preparing ADAM15. As a result, the inventors found that microvesicular ADAM15 could be very effectively prepared when exosomes were released through treatment with a protein kinase C (PKC) activator, and then completed the present disclosure.

Accordingly, an aspect of the present disclosure is to provide a method for preparing microvesicular ADAM15 from mammalian cells.

Another aspect of the present disclosure is to provide a method for inducing microvesicular ADAM15 generation, the method including a PKC activator.

Still another aspect of the present disclosure is to provide a method for preparing microvesicular ADAM15.

Still another aspect of the present disclosure is to provide a method for inducing microvesicular ADAM15 generation, the method including an immune adjuvant.

Other purposes and advantages of the present disclosure will become clarified by the following detailed description of invention, claims, and drawings.

### Technical Solution

In accordance with an aspect of the present disclosure, there is provided a method for preparing microvesicular ADAM15 from mammalian cells, the method including:

(a) activating PKC of isolated mammalian cells; and,

(b) isolating exosomes containing ADAM15 from the mammalian cells.

The present inventors investigated a biological importance of ADAM15, as an exosomal component, in the tumor suppression mechanism associated with the immune function of ADAM15, and then endeavored to develop a method for preparing ADAM15. As a result, the inventors found that microvesicular ADAM15 could be very effectively prepared when exosomes were released through treatment with a PKC activator.

As used herein, the term “microvesicular (or exosomal) ADAM 15” refers to ADAM 15 positioned on surfaces of exosomes.

According to the present disclosure, mammalian cells are treated with a PKC activator.

Preferably, in step (a), the PKC activator used to treat the mammalian cells may be phorbol-12-myristate-13-acetate (PMA), 1-oleoyl-2-acetylgylcerol, 1-stearoyl-2-arachidonoyl-sn-glycerol, 1,2-didecanoylglycerol, 1,2-dioctanoyl-sn-glycerol, (2S,5S)-8-decylbenzolactam V, 6-(N-decylamino)-4-hydroxymethylindole, 7-octylindolactam V, 1,2-di-O-octanoyl-3-O-β-D-galactopyranosyl-rac-glycerol, 12-deoxyphorbol 13-phenylacetate, 1-oreoyl-2-acetyl-sn-glycerol (OAG), 1-oreoyl-2-O-acetyl-3-β-D-galactopyranocyl sn glycerol, 1-stearoyl-2-linoleoyl-sn-glycerol, 8-hydroxy-[S-(E,Z,Z,Z)]-5,9,11,14-eicosatetraenoic acid, arachidonic acid, cholesterol 3-sulfate, oleic acid, phorbol 12,13-diacetate (PDA), phorbol 12,13-dibutyrate (PDBu), phorbol 12,13-didecanoate (PDD), 4α-phorbol-12,13-didecanoate, L-α-phosphatidylinositol-3,4-bisphosphate, L-α-phosphatidylinositol-4,5-bisphosphate, L-α-phosphatidyl inositol-3,4,5-triphosphate, 1-stearoyl-2-arachidonoyl-sn-glycerol, 5-chloro-N-heptyl-naphtalene-1-sulfonamide, resiniferatoxin (RTX), resiniferonol 9,13,14-ortho-phenylacetate (ROPA), 12-deoxyphorbol 13-phenylacate 20-acetate, indolactam V, 20-dibenzoate, phorbol 12,13-dihexanoate, phorbol-12,13-didecanoate, 1-hexylindolactam-V10, 6,11,12,14-tetrahydroxy-abieta-5,8,11,13-tetraene-7-one (coleon U), 8-octyl-benzolactam-V9, acetyl-L-carnitine, chloroform, retinoic acid, or phorbol ester 12-O-tetradecanoyl-phorbol-13-acetate. PMA is more preferably used.

As used herein, the term “mammalian cells” refers to cells isolated from mammalian tissues or tumors for in vivo experiment. The mammalian cells may be isolated as monocytes from tissues or tumors by digestion of enzymes, such as collagenase, trypsin, or pronase. Cells that are isolated from tissues or tumors and directly cultured are called primary cells. The primary cells have a restricted lifetime unless they are isolated from tumors. Established cells or immortalized cells can indefinitely proliferate through artificial operation such as expression of the telomerase gene, or random mutation.

Preferably the mammalian cells may be human cells.

As used herein, the term “human cells” refers to cells derived from human tissues or tumors.

Preferably, the mammalian cells may be normal cells or tumor cells.

Preferably, the cells may be immune cells.

As used herein, the term “immune cells” refers to cells involved in immune responses, which are hematopoietic-derived cells. The immune cells include lymphocytes such as B cells and T cells, natural killer cells, myeloid cells (e.g., monocytes, macrophages, esosinophils, mast cells, and basophils), and granulocytes.

Preferably, the immune cells may be macrophages. The macrophages are leukocytes differentiated from monocytes. The macrophages are involved in non-specific defense response (innate immunity) as well as specific immune response (adaptive immunity). The macrophages show phagocytosis to remove cellular debris and pathogens, and stimulate lymphocytes and other immune cells through defenses against pathogens.

According to the present disclosure, PMA increases the release of microvesicular ADAM 15, and binds to integrin αvβ3 to inhibit the interactions of integrin αvβ3 with vitronectin and fibronectin. When the interactions of the integrin αvβ3 with vitronectin and fibronectin are inhibited, adhesion, proliferation, and migration of cells are prevented, resulting in suppressing tumor growth. In addition, microvesicular ADAM 15 does not induce apoptosis.

In accordance with another aspect of the present disclosure, there is provided a method for inducing microvesicular ADAM15 generation, the method including a PKC activator.

Preferably, the PKC activator may be phorbol-12-myristate-13-acetate (PMA), 1-oleoyl-2-acetylgylcerol, 1-stearoyl-2-arachidonoyl-sn-glycerol, 1,2-didecanoylglycerol, 1,2-dioctanoyl-sn-glycerol, (2S,5S)-8-decylbenzolactam V, 6-(N-decylamino)-4-hydroxymethylindole, 7-octylindolactam V, 1,2-di-O-octanoyl-3-O-β-D-galactopyranosyl-rac-glycerol, 12-deoxyphorbol 13-phenylacetate, 1-oreoyl-2-acetyl-sn-glycerol (OAG), 1-oreoyl-2-O-acetyl-3-β-D-galactopyranocyl sn glycerol, 1-stearoyl-2-linoleoyl-sn-glycerol, 8-hydroxy-[S-(E,Z,Z,Z)]-5,9,11,14-eicosatetraenoic acid, arachidonic acid, cholesterol 3-sulfate, oleic acid, phorbol 12,13-diacetate (PDA), phorbol 12,13-dibutyrate (PDBu), phorbol 12,13-didecanoate (PDD), 4α-phorbol-12,13-didecanoate, L-α-phosphatidylinositol-3,4-bisphosphate, L-α-phosphatidylinositol-4,5-bisphosphate, L-α-phosphatidyl inositol-3,4,5-triphosphate, 1-stearoyl-2-arachidonoyl-sn-glycerol, 5-chloro-N-heptyl-naphtalene-1-sulfonamide, resiniferatoxin (RTX), resiniferonol 9,13,14-ortho-phenylacetate (ROPA), 12-deoxyphorbol 13-phenylacate 20-acetate, indolactam V, 20-dibenzoate, phorbol 12,13-dihexanoate, phorbol-12,13-didecanoate, 1-hexylindolactam-V10, 6,11,12,14-tetrahydroxy-abieta-5,8,11,13-tetraene-7-one (coleon U), 8-octyl-benzolactam-V9, acetyl-L-carnitine, chloroform, retinoic acid, or phorbol ester 12-O-tetradecanoyl-phorbol-13-acetate, but it not limited thereto.

According to a preferable embodiment of the present disclosure, step (b) microvesicles are isolated through density gradient fraction and ultracentrifugation.

In accordance with still another aspect of the present disclosure, there is provided a method for preparing microvesicular ADAM15, the method including:

(a) incubating microphages to generate exosomes containing ADAM15; and

(b) isolating the exosomes containing ADAM15.

Preferably, the method may further include, prior to step (a), differentiating monocytes into the macrophages, and more preferably, the differentiating may be conducted by using a PKC activator.

Preferably, the macrophages may be cells treated with an immune adjuvant, and more preferably, the immune adjuvant is liposome, lipopolysaccharide (LPS), and immunostimulatory oligonucleotide. The immunostimulatory nucleotide (e.g., CpG oligonucleotide) used herein includes any immunostimulatory nucleotide known in the art. The immunostimulatory oligonucleotide may be, for example, a particular palindrome forming a hairpin secondary structure, Cpg motif, CpT motif, multiple G-domains, or other known immunostimulatory sequences (ISS). For example, the immunostimulatory oligonucleotide used herein includes immunostimulatory oligonucleotides disclosed in US Patent Application No. 20080045473, and WO 2006/063152 and WO 1998/18810. As used herein, the term ‘CpG oligonucleotide’ refers to a nucleotide sequence that contains at least two unmethylated or methylated cytosine-guanine sites to activate immune responses. Synthetic oligonucleotides containing CpG motif (GpG-ODNs) activate various kinds of immune cells including microphages, dentric cells, NK cells, and B lymphocytes, thereby exhibiting immune-enhancing effects similar to direct immune-promoting effects by natural bacterial DNA.

In accordance with still another aspect of the present disclosure, there is provided a method for inducing microvesicular ADAM15 generation from macrophages, the method including an immune adjuvant.

Preferably, the immune adjuvant is liposome, lipopolysaccharide (LPS), or immunostimulatory oligonucleotide.

### Advantageous Effects

Features and advantages of the present disclosure are summarized as follows:

(a) The present disclosure can provide a method for preparing microvesicular ADAM15 from mammalian cells.

(b) The present disclosure can provide a novel ADAM15-mediated mechanism of cellular processes through investigation on functions of microvesicular ADAM15.

(c) The present disclosure can provide new anti-cancer agents using microvesicular ADAM15 that suppresses adhesion, proliferation, and mitigation of tumor cells.

### DESCRIPTION OF DRAWINGS

FIG. 1 verifies that ADAM15 is released into the extracellular space. FIG. 1A shows western blotting analysis of conditioned medium with antibody against ADAM15 extracellular domain. MCF-7 cells were incubated with 5 ng/Ml PMA in serum-free RPMI 1640 medium. P indicates pro-ADAM15 and M indicates mature ADAM15. In the results of the lower panel, data are represented as the mean±standard deviation of four experiments. *P<0.01; Student's t test. FIG. 1B shows western blotting analysis of cell lysates using the indicated antibodies. Cells were treated as described in the drawing. Arrows indicate pro-ADAM15 (P) and mature ADAM15 (M), respectively. FIG. 1C shows analysis of ADAM15 mRNA in MCF-7 cells. The results show semi-quantitative RT-PCR analysis of ADAM15 mRNA in MCF-7 cells treated with 5 ng/Ml PMA for the indicated times.

FIG. 2 verifies that released ADAM15 is associated with exosomes, and exosome-mediated ADAM15 is secreted by PMA-stimulated PKC activation. FIG. 2A shows western blotting analysis. Conditioned medium of MCF-7 cells treated with or without 5 ng/Ml PMA for 24 h was subjected to sequential centrifugation as follows: 300×g (P1), 1,200×g (P2), 10,000×g (P3), 100,000×g (P4), and 100,000×g after filtering through a 0.22-μm pore size filter (P4′). Pellets from each stage were subjected to western blotting analysis using an anti-ADAM15 cytoplasmic domain antibody. Arrows indicate pro-ADAM15 (P) and mature ADAM15 (M), respectively. FIG. 2B shows western blotting analysis of the fractions after sucrose gradient fractionation using the indicated antibodies. Isolated microvesicles from conditioned medium of MCF-7 cells treated with 5 ng/Ml PMA were subjected to sucrose gradient centrifugation. Each fraction was concentrated prior to SDS-PAGE. FIG. 2C shows effects of PMA stimulation on the secretion of exosome-associated ADAM15 in various cancer cell types. After ultracentrifugation at 100,000×g, the pellets were analyzed by immunoblotting with antibody to ADAM15 cytoplasmic domain. FIG. 2D shows effects of PKC inhibitor on the secretion of exosome-associated ADAM15. MDAH-2774 cells were treated with PMA and indicated concentrations of calphostin C for 24 h. Isolated exosomes were analyzed by immunoblotting with ADAM15 antibody. FIG. 2E shows flow cytometric analysis of cell- and exosomal-surface expression of ADAM15. MCF-7 cells were treated with or without 5 ng/Ml PMA for 24 h. The percent changes in fluorescence intensity associated with the presence of ADAM15 are shown.

FIG. 3 verifies that ADAM15-rich exosomes have a high binding affinity for integrin αvβ3 and prevents interactions between integrin αvβ3 and vitronectin. FIG. 3A shows analysis of various exosomes generated from HEK-293F cells expressing control vector (Exo-CON), ADAM15 (Exo-WT), ADAM15 D66E mutant (Exo-D66E), and ADAM15 E350A mutant (Exo-E350A). FIGS. 3B and 3C show immunoblotting analysis. Prior to exosome isolation, transfected cells were stimulated with or without 5 ng/Ml PMA for 24 h, and the isolated exosomes were analyzed by immunoblotting analysis with the indicated antibodies. The exosomes were incubated with purified integrin αvβ3 (FIG. 3B) or α5β1 (FIG. 3C), followed by ultracentrifugation. The precipitates were analyzed by immunoblotting analysis. In the graphs, the immunoblotting results are represented by the relative intensity. FIG. 3D shows effects of various exosomes on the binding of integrin αvβ3 to vitronectin. The exosomes were obtained from HEK-293F cells expressing control vector (Exo-CON), ADAM15 (Exo-WT), ADAM15 D66E mutant (Exo-D66E), and ADAM15 E350A mutant (Exo-E350A). Exosomes were incubated with purified integrin αvβ3 on a vitronectin-coated plate, and bound integrins were detected by anti-integrin αvβ3 antibody. In the results, data are represented as the mean±standard deviation of six experiments. FIG. 3E shows effects of various exosomes on cell adhesion to vitronectin. The exosomes were obtained from control vector (Exo-CON)-, ADAM15 (Exo-WT)-, ADAM15 D66E mutant (Exo-D66E)-, and ADAM15 E350A mutant (Exo-E350A)-transfected HEK-293 cells. MDAH-2774 cells were treated with exosomes on a vitronectin-coated plate. After 2-h incubation, the number of bound cells was determined by MTT assay (OD: 540 nm). In the results, graph, data are represented as the mean±standard deviation of six experiments. The right panel represents the level of plasma membrane-associated integrin αvβ3 in MDAH-2774 cells. *P<0.05 and **P<0.01; Student's t test).

FIG. 4 verifies that exosomal ADAM15 derived from transfected cells reduces vitronectin- and fibronectin-induced cell proliferation and migration. FIG. 4A shows effects of exosomal ADAM15 on MCF-7 cell proliferation. Cells (0.5×10⁴) were treated with 0.5 μg exosomes in RPMI 1640 medium containing 1% Fetal Bovine Serum (FBS) on 96-well plates coated with 1% BSA (control), fibronectin (10 μg/Ml), or vitronectin (8 μg/Ml). Exosomes were isolated from vector (Exo-CON)- or ADAM15 (Exo-A15)-transfected 293F cells stimulated with or without PMA. After 48-h incubation, cell number was determined by MTT assay (OD: 540 nm). FIG. 4B shows effects of exosomal ADAM15 on the proliferation of MDAH-2774 (2774) and NCI-460 (460) cells. In the results, data are represented as the mean±standard deviation of three experiments. FIG. 4C shows functional effects of the RGD motif in the anti-proliferative effect of exosomal ADAM15. MCF-7 cells were treated with control exosomes (Exo-CON) or exosomes containing ADAM15 (Exo-WT), ADAM15 R66E (Exo-R66E), or ADAM15 E350A (Exo-E350A) on 96-well plates coated with either vitronectin or fibronectin. Cell proliferation was determined by MTT assay. In the results, data are represented as the mean±standard deviation of three experiments. FIG. 4D shows effects of exosomal ADAM15 on cell migration. MDAH-2774 (2774) or NCl-460 (460) cells pre-incubated with exosomes were placed in trans-well chambers coated with either vitronectin or fibronectin. The number of migrated cells was counted under a light microscope. In the graph, data are represented as the mean±standard deviation of four experiments. FIG. 4E verifies that exosomal ADAM15 does not induce apoptosis. MDAH-2774 cells were incubated with exosomes on vitronectin-coated plates. After 48-h incubation, apoptotic cells were detected by TUNEL assay under a fluorescence microscope. Cells treated with DNase served as a positive control for apoptosis. *P<0.05 and **P<0.01 (Student's t test).

FIG. 5 verifies functional significance of exosomal ADAM15 derived from tumor cells through in vitro and in vivo experiments. FIG. 5A shows electron microscopy assay and immunoblotting analysis of exosomes. Exosomes were purified from MDAH-2774 cells (Exo) or PMA-stimulated MDAH-2774 cells (Exo-PMA). The isolated exosomes were confirmed by electron microscopy and immunoblotting analysis. The scale bar indicates 100 nm. FIG. 5B shows effects of exosomal ADAM15 derived from tumors on cell proliferation. MDAH-2774 cells (0.5×10⁴) were treated with 0.5 μg exosomes on 96-well plates coated with vitronectin (8 μg/Ml) in the presence or absence of ADAM15 extracellular domain antibody. After 48-h incubation, cell number was determined by MTT assay (OD: 540 nm). In the result, data are represented as the mean±standard deviation of five experiments. FIG. 5C shows effects of exosomal ADAM15 derived from tumors on cell migration. MDAH-2774 cells preincubated with exosomes were placed on transwell chambers coated with vitronectin. The number of migrated cells was counted by light microscopy. Data represent the mean and standard deviation of five experiments. FIG. 5D shows results of In vivo xenograft tumorigenicity. Exosomes were isolated from MDAH-2774 cells (Exo) or PMA-stimulated MDAH-2774 cells (Exo-PMA). MDAH-2774 cells with 20 μg exosomes was subcutaneously coinjected with 20 μg exosomes into mice. For functional blocking of exosomal ADAM15, exosomes were pre-incubated with ADAM15 antibody (Exo-PMA-Ab) prior to injection. The results show images and volumes of tumors after 20 days. *P<0.05 and **P<0.01 (Student's t test).

FIG. 6 verifies that exosomal ADAM15 is released from human macrophages. FIG. 6A shows western blotting analysis of THP-1 exosomes and cell lysates. PMA-differentiated THP-1 cells were washed, and further cultured for 2-4 days. THP-1 monocytes and differentiated macrophages (THP-1 Mφ) were incubated in serum-free medium for 24 h. Exosomes were isolated from the conditioned medium, followed by western blotting analysis. Arrows indicate pro-ADAM15 and mature ADAM15, respectively. FIG. 6B shows Fluorescence Activated Cell Sorting (FACS) analysis of THP-1 cells. THP-1 monocytes (THP-1 Mo) and differentiated macrophages (THP-1 Mφ) were double-stained with CD11b and ADAM15 antibodies. FIG. 6C shows western blotting analysis of exosomes derived from primary cells. Primary human monocytes (Primary Mo) and differentiated macrophages (Primary Mφ) were incubated in serum-free medium for 16 h. In the graph of the lower panel, data are represented as the mean±standard deviation of three experiments. *P<0.05 and **P<0.01 (Student's t test). FIG. 6D shows FACS analysis of primary human monocytes and differentiated macrophages.

FIG. 7 verifies that macrophage-derived ADAM15 exosomes have in vitro and in vivo tumor-suppressive functions. FIGS. 7A and 7B show effects of differentiated macrophage-derived ADAM15 exosomes on tumor growth and migration. Exosomes were isolated and treated from THP-1 monocytes (Exo-Mo) and differentiated macrophages (Exo-Mφ). Effects of these exosomes on MDAH-2774 cell growth and migration were examined in the presence or absence of an ADAM15 ectodomain-specific antibody. In the results, data are represented as the mean±standard deviation of six experiments. FIG. 7C shows effects of macrophage-derived exosomes on microphage proliferation and migration. In the results, data are represented as the mean±standard deviation of six experiments. FIG. 7D shows In vivo xenograft tumorigenicity. MDAH-2774 cells with or without macrophage exosomes were coinjected subcutaneously into mice, followed by measurement of tumor volumes. For functional blocking of exosomal ADAM15, microphage exosomes were pre-incubated with ADAM15 antibody (Exo-Mφ-Ab), prior to injection. *P<0.05 and **P<0.01 (Student's t test).

FIG. 8 verifies that exosomal ADAM15 does not induce apoptosis. MCF-7 cells were incubated with control exosomes (Exo-CON) or ADAM15-rich exosomes (Exo-A15) on fibronectin- or vitronectin-coated 96-well plates. After 48-h incubation, apoptotic cells were detected by TUNEL assay. Cells treated with DNase served as a positive control for apoptosis.

FIG. 9 verifies that the release of exosomal ADAM15 is further induced by LPS stimulation in differentiated THP-1 macrophages. FIG. 9A shows western blotting assay of exosomes and cell lysates. THP-1 cells were incubated with PMA in serum-free medium for 24 h. Arrows indicate pro-ADAM15 and mature ADAM15, respectively. Total protein bands of exosomes were shown by Coomassie blue staining (middle penal). In the graph of the right panel, data are represented as the mean±standard deviation of three experiments. FIG. 9B shows western blotting analysis of exosomes. PMA-differentiated THP-1 cells were washed with PBS and further cultured for 2˜3 days. THP-1 macrophages were incubated with LPS in serum-free medium for 18 h. Exosomes were isolated from conditioned media. Arrows indicate pro-ADAM15 and mature ADAM15, respectively. Total protein bands of exosomes were shown by Coomassie blue staining (middle penal). The right panel represents the mean and standard deviation of three experiments. FIG. 9C shows western blotting analysis of exosomes. THP-1 cells were treated with or without PMA and/or LPS in serum-free medium for 18 h. Exosomes were isolated from conditioned media. The arrow indicates mature ADAM15. In the graph of the right panel, data are represented as the mean±standard deviation of three experiments. *P<0.05 and **P<0.01 (Student's t test).

FIG. 10 verifies that plasma membrane-associated ADAM15 does not affect cell-ECM interactions. FIG. 10A shows effects of ADAM15 expression on cell adhesion to fibronectin, vitronectin, collagen I, and collagen IV. MCF-7 cells were transfected with control (CON), ADAM15 (A15), control siRNA (si-CON), or ADAM15 siRNA (si-A15) prior to cell adhesion assay. Expression levels of ADAM15 were confirmed by immunoblotting and FACS analysis. Attached MCF-7 cells (5×10⁴) were seeded on 96-well plates coated with fibronectin (FN), vitronectin (VN), collagen I (COL-I), and collagen IV (COL-IV) and incubated for 2 h. The number of attached cells was determined by MTT assay. In the results, data are represented as the mean±standard deviation of three experiments. FIG. 10B shows flow cytometric analysis of cell surface expression of integrins αvβ3 and α5β1 in transfected MCF-7 cells. A sample lacking primary antibody was used as a control.

### BEST MODEL

Hereinafter, the present disclosure will be described in detail with reference to examples. These examples are only for illustrating the present disclosure more specifically, and it will be apparent to those skilled in the art that the scope of the present disclosure is not limited by these examples.

### EXAMPLES

### 1. Materials and Methods

### 1.1. Materials

Mouse monoclonal anti-ADAM15 extracellular domain antibody (MAB935) was purchased from R&D Systems (Minneapolis, Minn.). Mouse monoclonal antibodies specific to integrin αvβ3 (MAB1976Z), integrin αv (MAB1930), integrin α5β1 (MAB1969), integrin β1 (MAB1965), integrin β3 (MAB1932), and glyceraldehyde 3-phosphate dehydrogenase (GAPDH) (MAB374), as well as rabbit polyclonal anti-ADAM15 cytoplasmic domain antibody (AB19036) were purchased from Chemicon (Temecula, Calif.). Mouse monoclonal anti-CD11b (FCMAB178P) was purchased from Millipore (Billerica, Mass.). Mouse monoclonal antibodies specific to CD9 (sc-13118), TSG101 (sc-7964), ADAM10 (sc-28358) and ADAM15 (sc-73686) were purchased from Santa Cruz Biotechnology (Santa Cruz, Calif.), and mouse monoclonal anti-E-cadherin (BD610181) was purchased from BD Biosciences (San Jose, Calif.). PMA (P1585) was purchased from Sigma-Aldrich (St. Louis, Mo.).

### 1.2. Expression Plasmids and Site-Directed Mutagenesis

The sequence of full-length ADAM15 (Accession number NM_{—}003815.3) was obtained from the National Center for Biotechnology Information. PCR was performed to amplify full-length ADAM15 cDNA from human fetal liver (Marathon cDNA, Clontech, Palo Alto, Calif.) using primers 5′-ATGCGGCTGGCGCTGCTCTGG-3′ and 5′-TCAGAGGTAGAGCGAGGACAC-3′. The PCR product was cloned into TOPO cloning vectors (Invitrogen, Carlsbad, Calif.) and sequenced. For the full-length ADAM15 expression plasmid, the open reading frame was digested with XhoI/BamH1 and then re-cloned into the pcDNA3.1/myc-His(−) B vector (Invitrogen) previously digested with XhoI/BamH1. All mutants used for the present study were generated by site-directed mutagenesis (Intron Biotechnology, Korea).

### 1.3. Cell Culture, Transfection, and MTT Assay

MCF-7 (human breast adenocarcinoma) (ATCC no. HTB-22, Manassas, Va.), NCI-H460 (human lung carcinoma) (ATCC no. HTB-177), and THP-1 (human monocyte) (ATCC no. TIB-202) cells were maintained in RPMI 1640 medium containing 10% fetal bovine serum (FBS). MDAH 2774 (human ovarian adenocarcinoma) (ATCC no. CRL-10303), A549 (human lung carcinoma) (ATCC no. CCL-185), SK-MEL-28 (human melanoma) (ATCC no. HTB-72), and HEK293F cells were maintained in Dulbecco's Modified Eagle Medium (DMEM) containing 10% FBS. Transfection with plasmids and small interfering RNAs (siRNAs) was performed using Lipofectamine 2000 according to the manufacturer's instructions (Invitrogen). Hygromycin B (Invitrogen) (500 μg/ml) was used to establish stable cell lines. For the determination of viable cell number, cells were incubated with 50 μg/ml MTT [3-(4,5-dimethylthiazol-2-yl)-2,5-diphenyltetrazolium bromide] for 2 h. Dimethyl sulfoxide (100 μl/well) was added to solubilize the crystals, and absorbance was measured at 595 nm.

### 1.4. Western Blotting

Cells were lysed in buffer containing 50 Mm Tris-HCl, pH 7.5, 5 mM EDTA, 150 mM NaCl, 1% Triton X-100, and protease inhibitor cocktail (Roche Applied Science, Indianapolis, Ind.) for 1 h at 4° C. and then centrifuged. The soluble portion of the lysate was used for western blotting, which was performed by separation of reduced or non-reduced samples on SDS-PAGE, followed by electroblotting to nitrocellulose membranes and detection of bound antibody by enhanced chemiluminescence (Amersham Biosciences, Pittsburgh, Pa.).

### 1.5. Isolation of Microvesicles by Sequential Centrifugation and Sucrose Density Gradient Fractionation

Cells were incubated with or without PMA in serum-free medium or medium containing 5% FBS for 24 h. Bovine microvesicles were removed from FBS by ultracentrifugation prior to use. The conditioned medium was sequentially centrifuged at 300×g for 10 min, 1,200×g for 20 min, and 10,000×g for 30 min to remove cell debris. The supernatants were then subjected to ultracentrifugation at 100,000×g for 1 h in a Beckman 70 Ti rotor. To remove the treated PMA completely, the pellets were washed by PBS. The microvesicular pellets were resuspended in 100-150 μl PBS and stored at −80° C. The supernatants were filtered through a 0.22-μm pore size filter (Millipore, Billerica, Mass.) prior to ultracentrifugation as previously described to remove shed vesicles from the microvesicles (Valadi et al., 2007). Protein concentration was measured by a modified Bradford assay (Bio-Rad Laboratories, Hercules, Calif.). Sucrose density gradient fractionation was performed as previously described with minor modifications to obtain exosomes (Gutwein et al., 2005). Microvesicles were resuspended in 0.25 M sucrose and loaded on the top of a step gradient containing layers of 2, 1.3, 1.16, 0.8, 0.5, and 0.25 M sucrose. The tubes were then ultracentrifuged at 100,000×g for 2.5 h in a Beckman SW 55 Ti rotor, and fractions with different densities were sequentially collected from the top of the tube and analyzed for exosomes containing ADAM15.

### 1.6. Electron Microscopy

Immunoelectron microscopy was performed as previously described with minor modifications (Hawari et al., 2004). Exosomes were loaded onto formvar carbon-coated nickel grids and fixed in 4% paraformaldehyde. Samples were incubated with a monoclonal antibody against the ADAM15 extracellular domain followed by 5 nm gold-conjugated goat anti-mouse antibody (Abcam). Images were acquired with a Transmission Electron Microscope (JEM-2010).

### 1.7. Flow Cytometry

Flow cytometric analysis of exosomes was performed as previously described with minor modifications (Thery et al., 2001). Exosome-coated beads were prepared by incubating 5 μg exosomes with 5 μg aldehyde/sulfate latex beads [4% (w/v) in 4 μm] in 100 μl PBS for 20 min and gentle shaking in 1 ml PBS for a further 2 h. The mixture was then treated with 100 mM glycine for 30 min and washed three times with PBS to stop the reaction. For the analysis of cell surface ADAM15, cells were fixed with 4% paraformaldehyde for 30 min and detached from 6-well plates using PBS containing 5 mM EDTA. Cells and exosome-coated beads were incubated with a monoclonal antibody against the ADAM15 extracellular domain at 4° C. for 2 h. The cells and exosome-coated beads were washed and further incubated with fluorescein isothiocyanate (FITC)-conjugated goat anti-mouse antibody (Chemicon) for 2 h. Flow cytometry was performed on a FACSCalibur (BD Biosciences), and data were analyzed using WinMDI software version 2.8 (The Scripps Research Institute, La Jolla, Calif.).

### 1.8. Exosome-Integrin Binding Assay

Exosomes (5 μg) were incubated with 100-200 ng purified integrin αvβ3 and α5β1 (R&D Systems) at 4° C. for 16 h in 1 ml PBS supplemented with 200 μM Mn²⁺. The incubated mixture was diluted with PBS to 5 ml followed by ultracentrifugation at 100,000×g for 1 h. The precipitates were analyzed by western blotting under non-reducing conditions.

### 1.9. Integrin-Vitronectin Binding Assay

Purified integrin αvβ3 (200 ng/ml) was incubated for 1.5 h in 100 μl serum-free RPMI 1640 medium with or without exosomes (5 μg/ml) on 96-well plates coated with vitronectin (8 μg/ml) (BD Biosciences). After washing four times with serum-free RPMI 1640 medium, bound integrin αvβ3 was incubated first with anti-integrin αv monoclonal antibody and subsequently with an HRP-conjugated goat anti-mouse antibody (Santa Cruz Biotechnology). The binding reactions were visualized with tetramethylbenzidine and measured at 450 nm.

### 1.10. Cell Migration Assay

Cell invasion assays were performed using transwell inserts with 6.5-mm diameter polycarbonate 8-μm microporous membranes (Costar, Cambridge, Mass.). The outer membranes of the transwell inserts were coated with vitronectin or fibronectin (1 μg/well). Cells (3.0×10⁴) preincubated in 100 μl serum-free medium with 2 μg exosomes were placed in the upper chamber. Then, 600 ill DMEM containing 10% FBS were added to the lower chamber. After 16 h, any cells remaining on the inner membrane were removed with a cotton swab. The transwells were fixed in 4% formaldehyde and stained with 10% Giemsa. Cell number was determined by light microscopy.

### 1.11. Xenografttumorigenesis Assay

Five-week-old female BALB/c nu/nu mice were obtained from Orient Bio Inc. (Seongnam, Korea) and maintained under specific, pathogen-free conditions. MDAH-2774 cells (0.5×10⁶) were injected into the flanks of mice with or without 20 μg exosomes. Tumor volumes were measured with a caliper, and the volumes were calculated according to the following formula: volume=0.52×length×width². Protocols were approved by the animal ethics committee of Yonsei University and carried out in accordance with established guiding principles for animal research.

### 1.12. Primary Human Monocyte/Macrophage Preparation

PBMCs were separated from the buffy coat fraction of healthy donor blood by Ficoll-Paque (GE healthcare) density gradient centrifugation. Human primary monocytes were isolated using a Monocyte Isolation Kit according to the manufacturer's recommendations (MiltenyiBiotec), yielding an average purity of 98%. Macrophages were obtained by culturing monocytes for 5 days in RPMI-1640 medium containing 20% FBS and 50 ng/ml M-CSF (R&D Systems).

### 1.13. Statistical Analysis

All experiments were repeated at least three times with similar results. Data are represented as the mean±standard deviation of n independent experiments. Statistical analysis was performed using an unpaired Student's t test. A p value less than 0.05 was considered statistically significant.

### 2. Results

### 2.1. ADAM15 is Released into the Extracellular Space

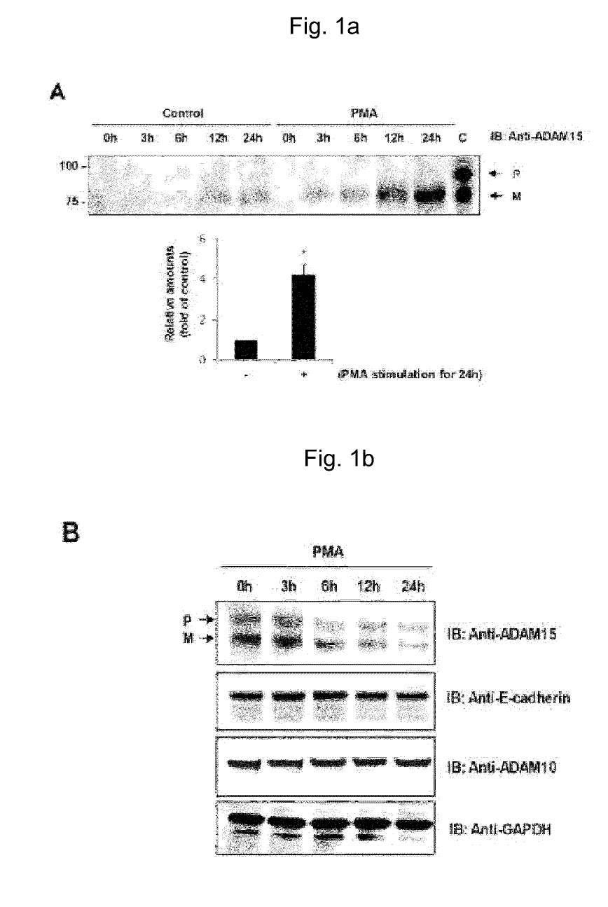

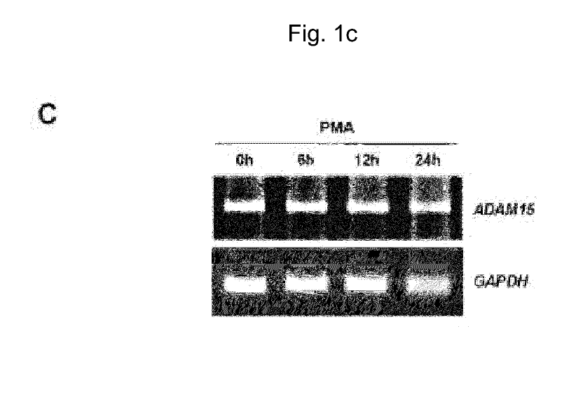

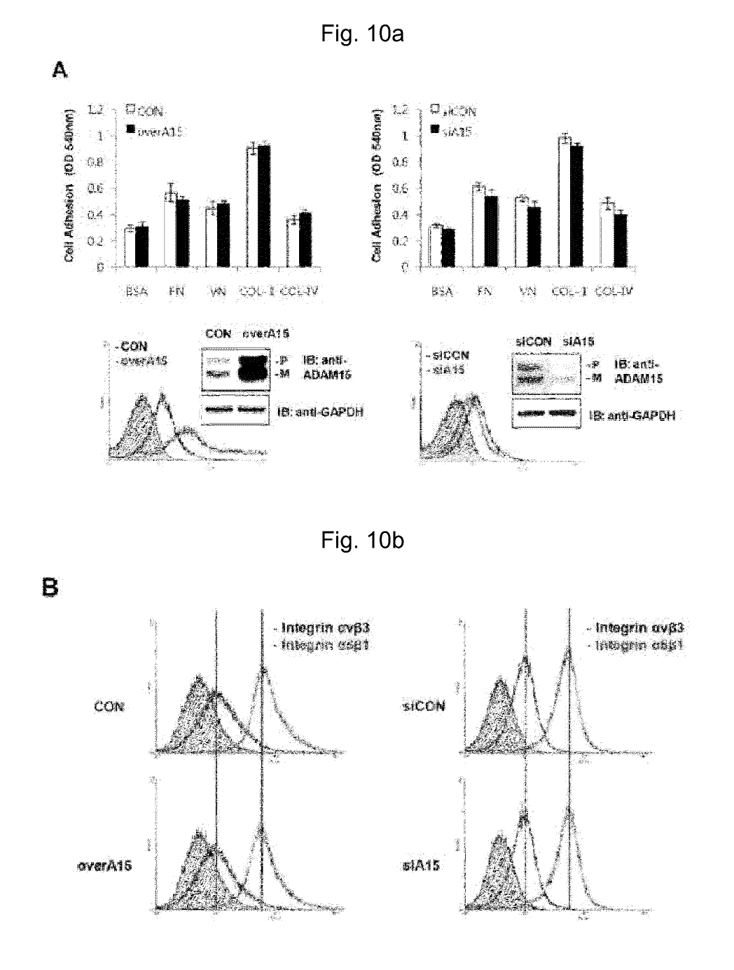

There are several studies showing that various membrane proteins are secreted into the extracellular space through ectodomain shedding or microvesicle release (van Kilsdonk et al., 2010). Here, we examined whether ADAM15 protein is released from human breast cancer MCF-7 cells into conditioned medium. Mature ADAM15 was detected in the conditioned medium by immunoblotting analysis with an antibody against ADAM15 extracellular domain (FIG. 1A). ADAM15 release was largely increased after stimulation with PMA, an activator of various membraneprotein-releasing events (Arribas et al., 1996; Hahn et al., 2003; Vecchi et al., 1996). In MCF-7 cells, both the pro and mature forms of ADAM15 were detected. Both forms decreased with time after PMA stimulation and were barely detectable after 24 hrs (FIG. 1B). However, other membrane proteins including ADAM10 and E-cadherin in MCF-7 cells were unchanged by PMA stimulation (FIG. 1B), indicating that ADAM15 is specifically decreased in the cells. The mRNA levels of ADAM15 after PMA stimulation were also assessed. As shown in FIG. 10, ADAM15 mRNA levels were unchanged by PMA, confirming that the release of plasma membrane-associated ADAM15 protein into the extracellular space, but not the synthesis of ADAM15 mRNA, is stimulated by PMA. We further investigated whether released ADAM15 was generated by ectodomain shedding. The mature form of ADAM15 was also detected in conditioned media by immunoblotting analysis with antibody specific for the ADAM15 cytoplasmic domain (data not shown), indicating that ADAM15 release is not associated with proteolytic cleavage of its extracellular domain.

### 2.2. Extracellular ADAM15 is Associated with Exosomes, and ADAM15-Rich Exosomes are Generated by PMA-Mediated PKC Activation

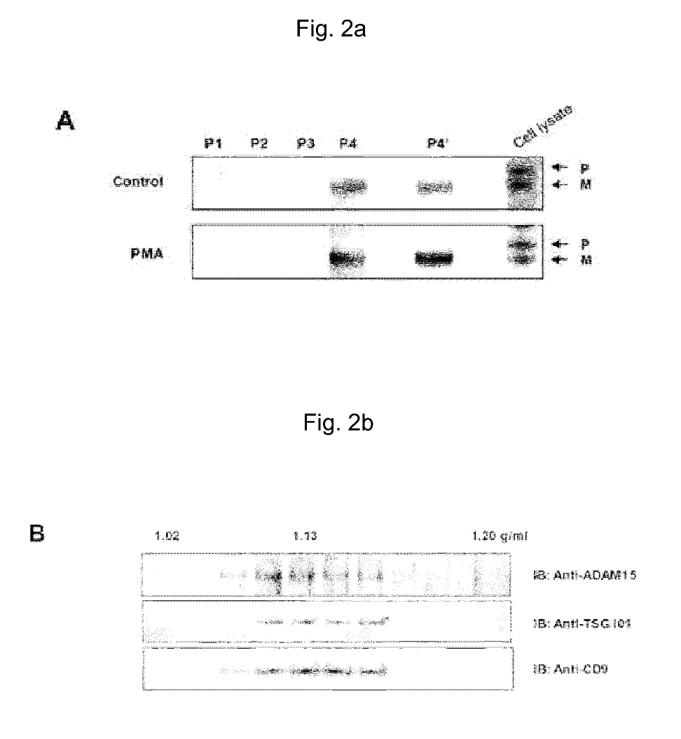

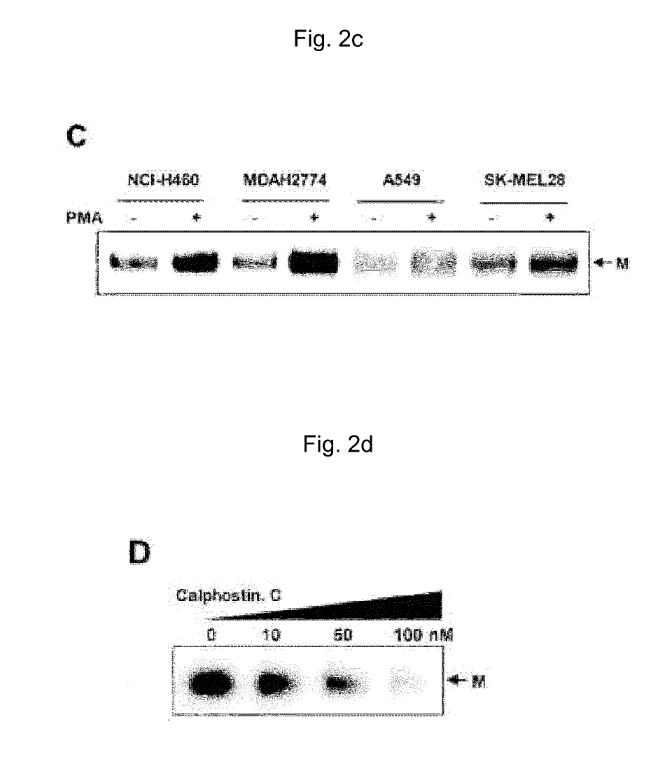

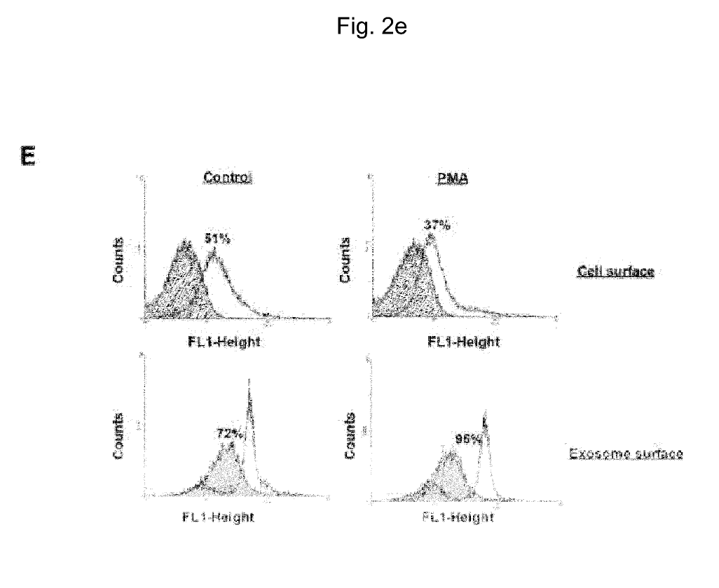

Because the released ADAM15 was not a shed form, we postulated that it might exist in microvesicles. Conditioned media of MCF-7 cells were analyzed by sequential centrifugation followed by immunoblotting analysis with anti-ADAM15 antibody. The mature form of ADAM15 was detected in the 100,000×g pellet containing microvesicles (FIG. 2A). As microvesicles consist of both shed vesicles and exosomes, it was examined which of these components contained ADAM15. Prior to ultracentrifugation, the conditioned medium was passed through a 0.22-μm pore size filter to remove shed vesicles (Valadi et al., 2007). As shown in FIG. 2A, ADAM15 was observed exclusively in the filtered pellet, suggesting that ADAM15 is associated with exosomes. The exosome-associated release of ADAM15 was further confirmed by continuous sucrose gradient ultracentrifugation. As shown in FIG. 2B, the released ADAM15 was detected together with exosomal markers CD9 and TSG101 in the range of 1.11 to 1.16 g/ml, indicating that released ADAM15 is an exosomal component. The amount of exosomal ADAM15 was increased by PMA stimulation (FIG. 2A, lower panel). Similar results were obtained with other cancer cell types (FIG. 2C), suggesting that this response is not cell type specific It was also examined whether the ADAM15 release is associated with PKC activation by PMA. As show in FIG. 2D, PMA-stimulated ADAM15 release was successfully blocked by calphostin C, a widely used PKC specific inhibitor (Linden and Connor, 1991), indicating that PKC activation is involved in the release process of ADAM15 When flow cytometry was performed to determine the amount of ADAM15 at the exosomal surface, PMA stimulation resulted in an increase in exosomal ADAM15 with a corresponding decrease in plasma membrane-associated ADAM15 (FIG. 2E), showing that ADAM15-rich exosomes are generated following PMA-mediated PKC activation.

### 2.3. Exosomal ADAM15 Binds to Integrin αvβ3

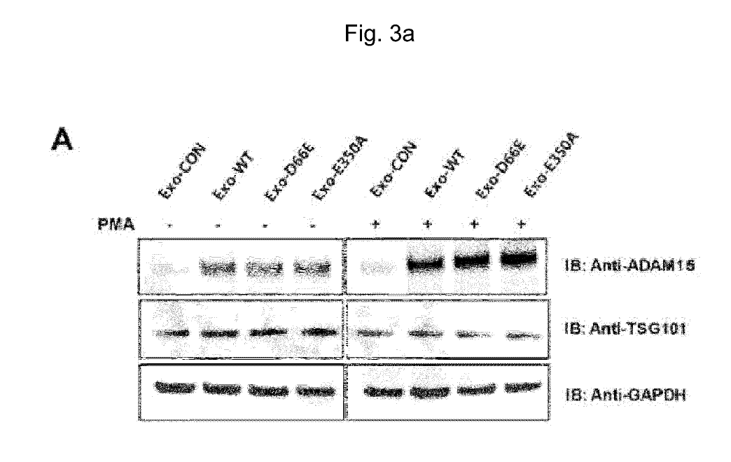

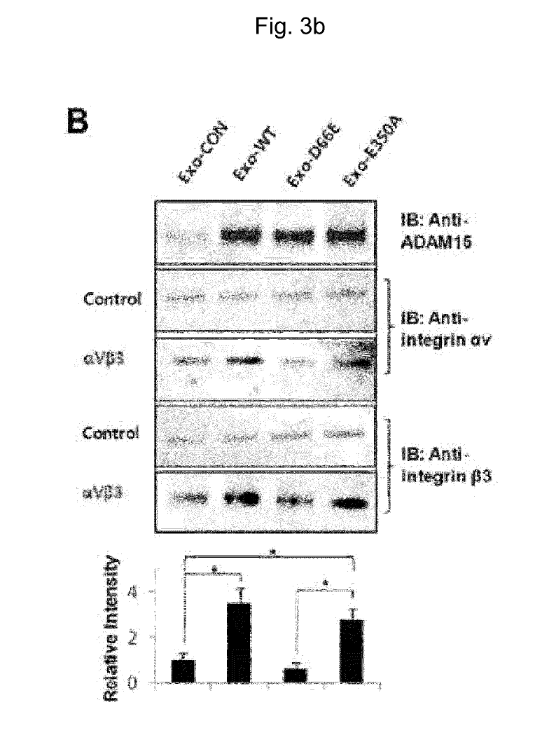

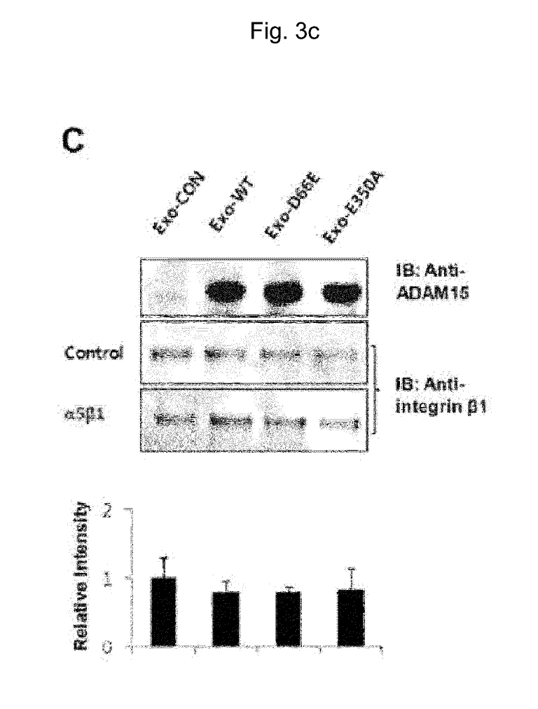

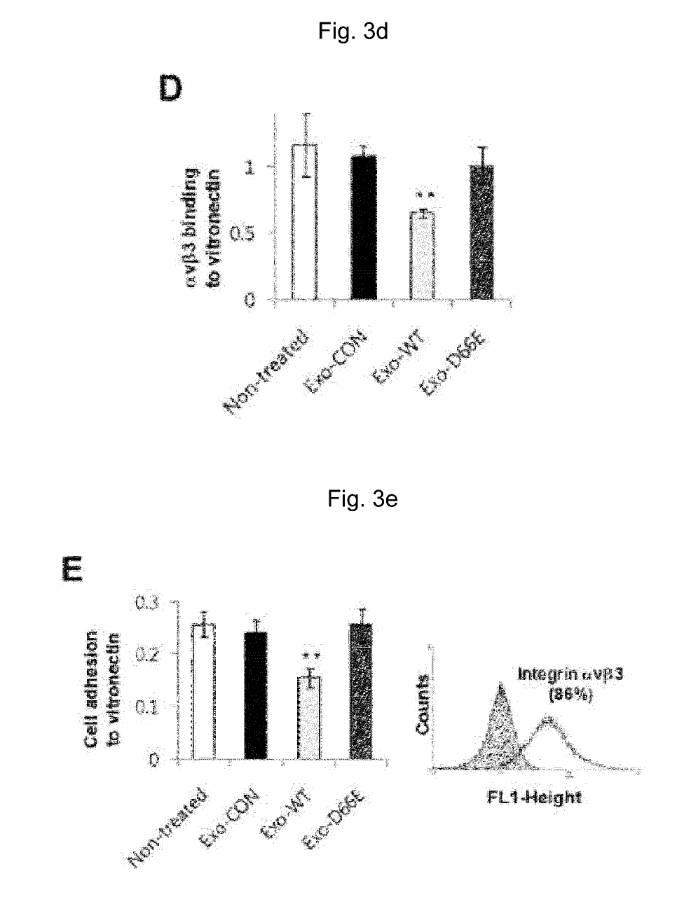

Exosomal surface molecules interact with their receptors on target cells and play key roles in many biological and pathological processes (Anand, 2010; Thery et al., 2009). The ADAM15 disintegrin-like domain interacts with integrins αvβ3 and α5β1 (Nath et al., 1999; Zhang et al., 1998). It was attempted to examine the binding affinities of exosomal ADAM15 for these integrins. For binding analyses, exosomes were obtained by sequential centrifugation from conditioned medium of HEK293F cells stably transfected with empty vector, ADAM15, or ADAM15 mutants. ADAM15 mutants had substituted amino acids in the RGD motifs of the ADAM15 disintegrin-like domain (D66E) and catalytic site of the metalloprotease domain (E350A), respectively. As shown in FIG. 3A, released exosomal ADAM15 was increased after transfection with ADAM15 and further exosomal enrichment of the protein was accomplished by PMA stimulation. When the ADAM15-rich exosomes were incubated with purified integrins αvβ3 and α5β1 and subjected to ultracentrifugation, the western blot analysis of the precipitates with anti-integrin antibodies showed an increased exosome binding affinity for integrin αvβ3 (FIG. 3B) but not for integrin α5β1 (FIG. 3C) compared to control exosomes. Furthermore, the exosome binding for integrin αvβ3 was abolished by mutating RGD in the disintegrin-like domain of ADAM15 to RGE (D66E), indicating an essential role of the RGD motif in this binding (FIG. 3B). Taken together, these experimental data indicate that exosomal ADAM15 specifically retains its binding affinity for integrin αvβ3 in an RGD-dependent manner.

### 2.4. Exosomal ADAM15 Inhibits Interaction of Integrin αvβ3 and Vitronectin

Integrin αvβ3 is involved in cell adhesion, migration, and proliferation through interactions with vitronectin and fibronectin (Brooks et al., 1994; Brooks et al., 1995; Landen et al., 2008) and that these interactions are abrogated by RGD-containing disintegrins (Chung et al., 2003; McLane et al., 2004). To observe whether the interaction between integrin αvβ3 and vitronectin is affected by exosomal ADAM15, recombinant integrin αvβ3 was incubated with various exosomes on vitronectin-coated plates. As shown in FIG. 3D, exosomal ADAM15 significantly inhibited the binding of recombinant integrin αvβ3 to vitronectin. In this context, exosomal ADAM15 inhibited the adhesion of MDAH-2774 cells, which abundantly express integrin αvβ3 (FIG. 3E, right panel), to vitronectin (FIG. 3E, left panel). These inhibitory effects disappeared after mutation of the ADAM15 RGD motif (D66E) (FIG. 3D and 3E). These experimental data clearly indicate that the RGD motif of exosomal ADAM15 is essential for inhibiting the interaction between integrin αvβ3 and vitronectin.

### 2.5. Exosomal ADAM15 Suppresses Vitronectin- and Fibronectin-Induced Cell Proliferation and Migration

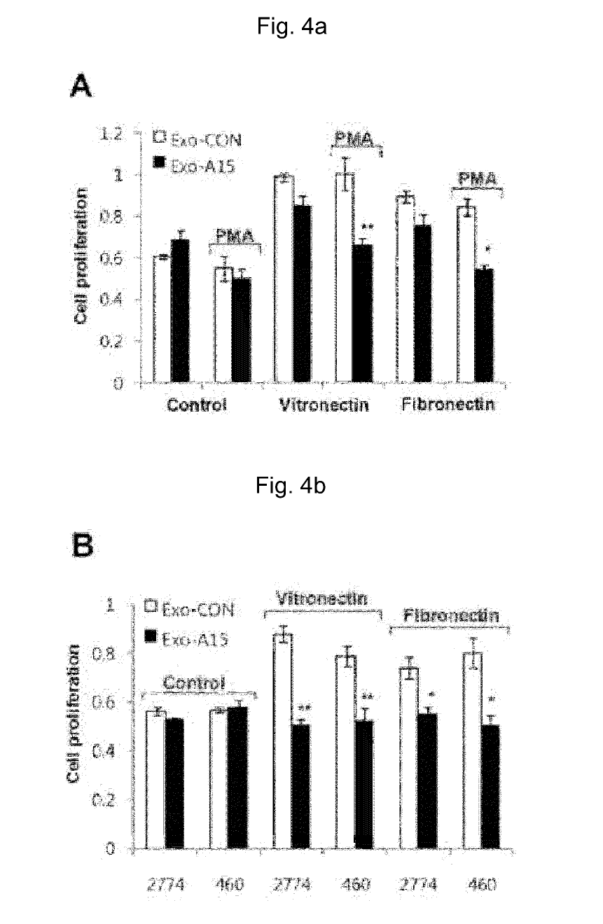

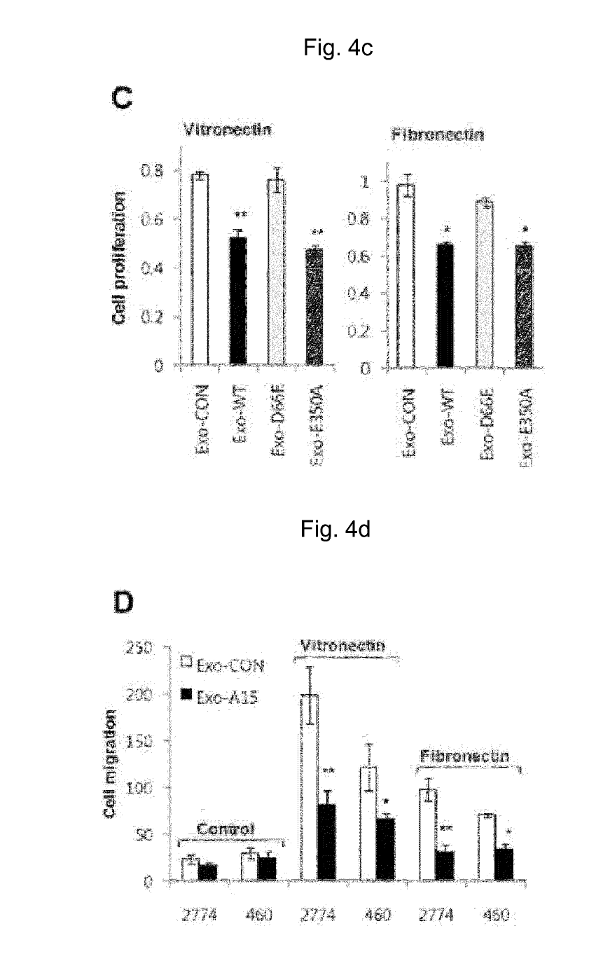

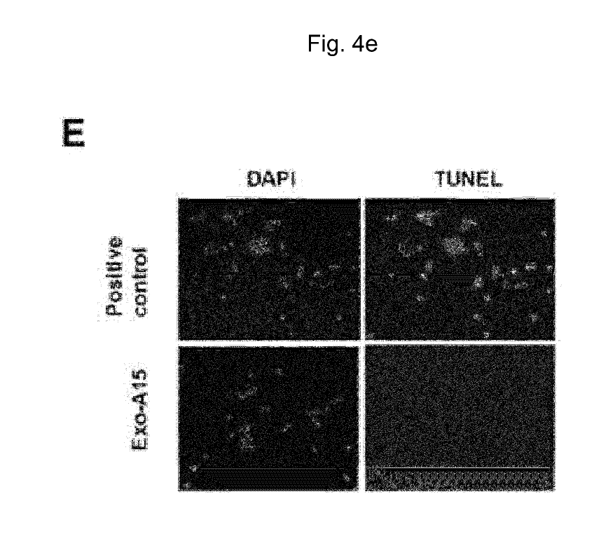

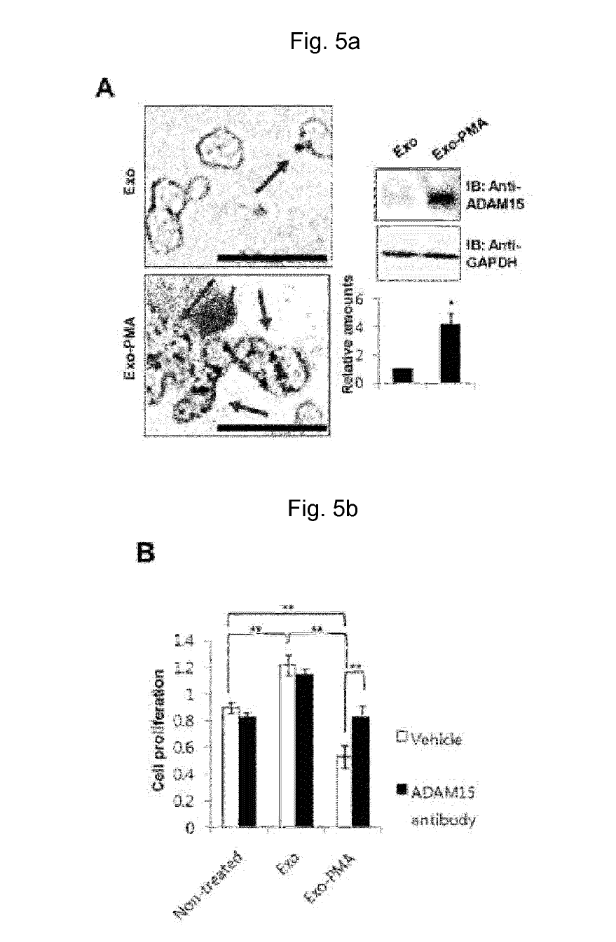

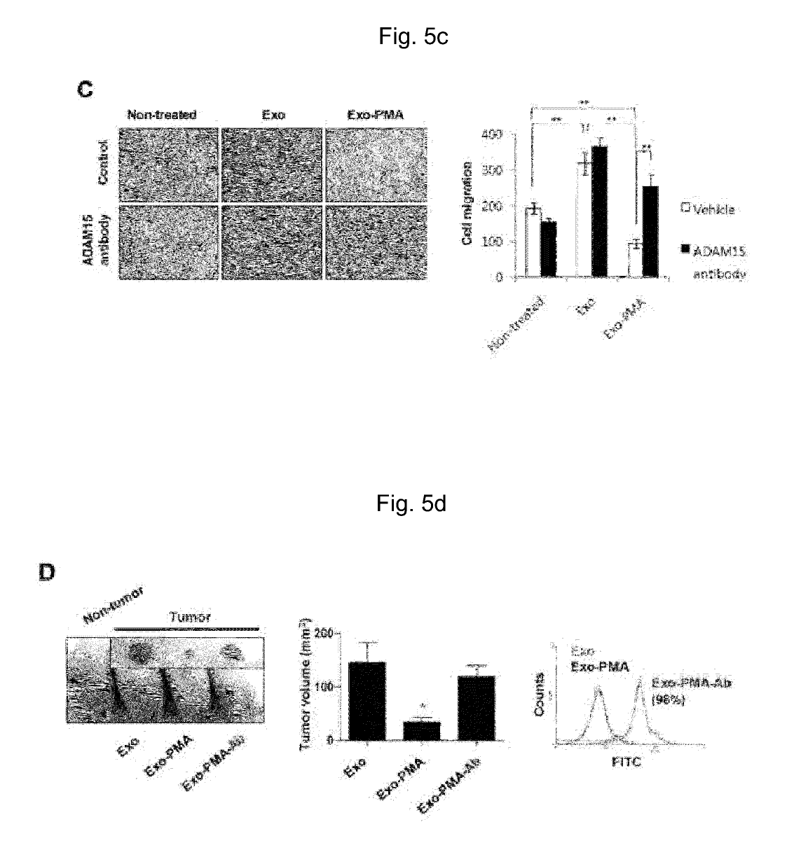

To examine the functional role of exosomal ADAM15, exosomal ADAM15 was isolated from ADAM15-transfected 293F cells with or without PMA stimulation. When MCF-7 cells were cultured on plates coated with vitronectin or fibronectin, the cell proliferation was increased on vitronectin and fibronectin compared to BSA control (FIG. 4A). Vitronectin- and fibronectin-induced cell proliferations were slightly reduced by exosomal ADAM15 isolated from unstimulated cells, and this suppressive effect was significantly enhanced by ADAM15-rich exosomes prepared from PMA-stimulated cells (FIG. 4A). Similar results were observed with other cancer cells, such as human lung cancer NCI-H460 and human ovarian cancer MDAH-2774 cells (FIG. 4B). To further explore the functional role of the RGD motif and the metalloprotease activity of ADAM15 in cell proliferation, ADAM15 D66E and catalytically inactive ADAM15 E350A mutants were employed. When cells were cultured in the presence of exosomes containing wild-type ADAM15 and catalytically inactive ADAM15 (E350A), cell proliferation was significantly reduced (FIG. 4C). In contrast, the ADAM15 D66E mutant did not affect the cell proliferation (FIG. 4C), indicating that the RGD motif containing the disintegrin-like domain, but not metalloprotease activity, is responsible for suppressing the proliferation. Exosomal ADAM15 also strongly inhibited both vitronectin- and fibronectin-induced cell migration (FIG. 4D), indicating that cancer progression may be regulated by exosomal ADAM15. It was then examined whether ADAM15-rich exosomes are able to induce apoptosis using the TUNEL assay. Immunofluorescence data showed no apoptotic processes in the cells treated with ADAM15-rich exosomes (FIG. 5E and FIG. S1). Taken together, these experimental data suggest that exosomal ADAM15 is primarily responsible for anti-tumor effects by inhibiting cell-extracellular matrix (ECM) interactions.

### 2.6. Tumor-Derived Exosomal ADAM15 Suppresses Cell Proliferation and Migration In Vitro and Tumor Growth In Vivo

To investigate the functional significance of tumor-derived exosomal ADAM15, tumor exosomes were isolated from MDAH-2774 cells stimulated with or without PMA. ADAM15 enrichment in isolated exosomes was verified by electron microscopy and immunoblotting analysis (FIG. 5A). When MDAH-2774 cell proliferation and migration assays were performed on vitronectin, ADAM15-rich exosomes were able to effectively reduce the cell proliferation and migration, and these suppressive effects were blocked by ADAM15 extracellular domain-specific antibody (FIGS. 5B and 5C). It was interesting to observe the enhanced cell proliferation and migration by exosomes derived from the non-stimulated tumor cells (FIGS. 5B and 5C). However, this stimulatory effect was not affected by ADAM15 antibody.

In a nude mouse model, ADAM15-rich exosomes were able to successfully suppress in vivo tumor growth compared to control exosomes (FIG. 5D, left and middle panels). To identify the functional role of exosomal ADAM15 in vivo, ADAM15-rich exosomes were preincubated with ADAM15 antibody and then subjected to ultracentrifugation followed by FACS analysis to examine the antibody blocking (FIG. 5D, right panel). As illustrated in FIG. 5D (left and middle panels), the antibody-blocked exosomes failed to suppress the in vivo tumor growth in the nude mouse model. These results imply the critical role of exosomal ADAM15 in tumor suppression mechanism.

### 2.7. Human Monocyte-Derived Macrophages Release Exosomal ADAM15

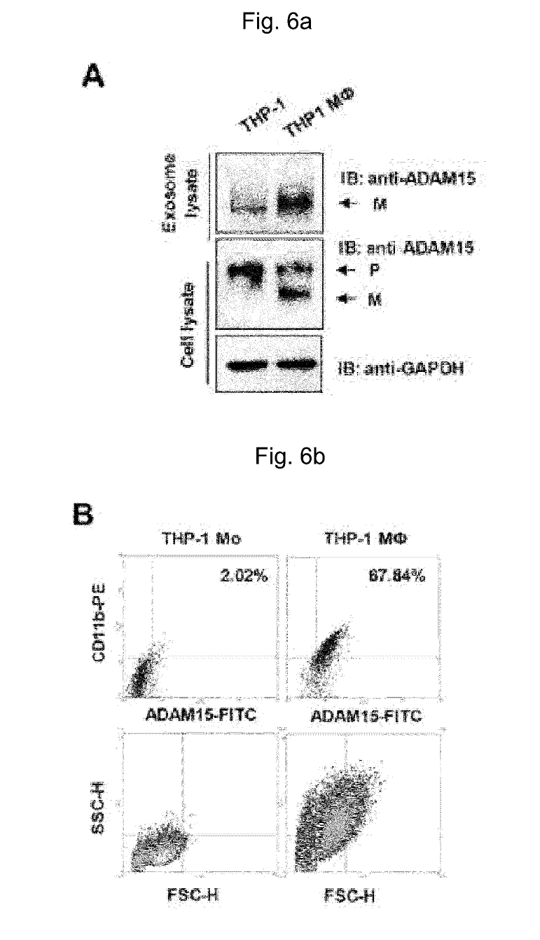

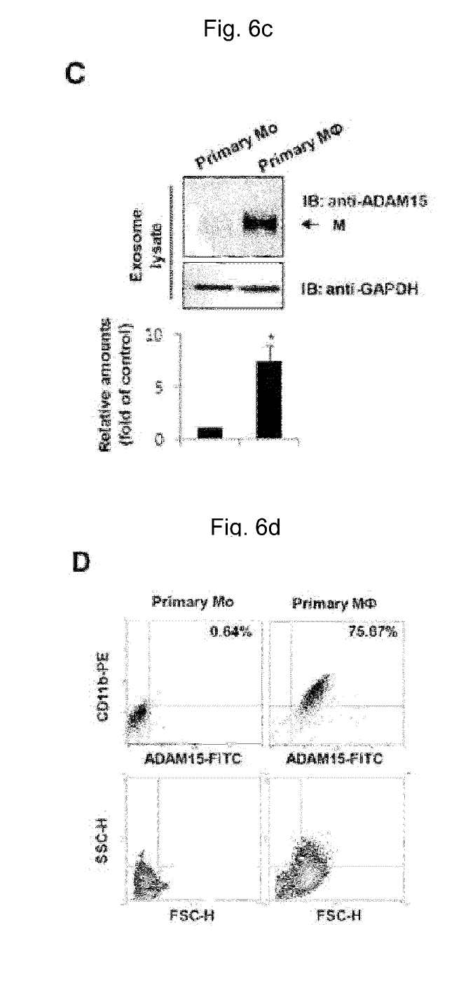

Based on the suppressive function of released exosomal ADAM15 in tumor growth, it was investigated whether tumor suppressive exosomal ADAM15 is released from human macrophages. First, THP-1 monocytes were differentiated into macrophages by PMA, a known stimulator of THP-1 differentiation into macrophages (Daigneault et al., 2010). Stimulation of THP-1 cells with PMA effectively increased the release of exosomal ADAM15, together with differentiation of the monocytes into macrophages as reflected by increased adherence (FIG. S2A). To fully differentiate THP-1 cells and exclude any direct effects of PMA, PMA-differentiated THP-1 cells were washed with PBS and further cultured for additional 2-3 days (Daigneault et al., 2010; Whatling et al., 2004). Fully differentiated THP-1 cells possessed macrophage features, increased levels of CD-11b and side scatter as verified by FACS analysis (FIG. 6B). As shown in FIGS. 6A and 6B, maturation and surface expression of ADAM15 were significantly increased, and exosomal ADAM15 was found to be continuously released in the differentiated macrophages. Release of exosomal ADAM15 was also enhanced by lipopolysaccharide (LPS), a known activator of anti-tumor immune responses (Hibbs et al., 1982), in the macrophages (FIGS. S2B and S2C), demonstrating that the release of exosomal ADAM15 is associated with monocyte/macrophage differentiation and activation.

The release of exosomal ADAM15 was further investigated in primary cultured human macrophages. Primary cultured human monocytes were isolated from buffy coats of normal blood donors and differentiated into macrophages (FIG. 6D). Likewise THP-1 macrophages, ADAM15 surface expression was also significantly increased in primary cultured human macrophages, and exosomal ADAM15 was abundantly released (FIGS. 6C and 6D), suggesting that the exosome release physiologically occurs in innate immune system.

### 2.8. Tumor Suppressive Function of Macrophage-Derived Exosomal ADAM15

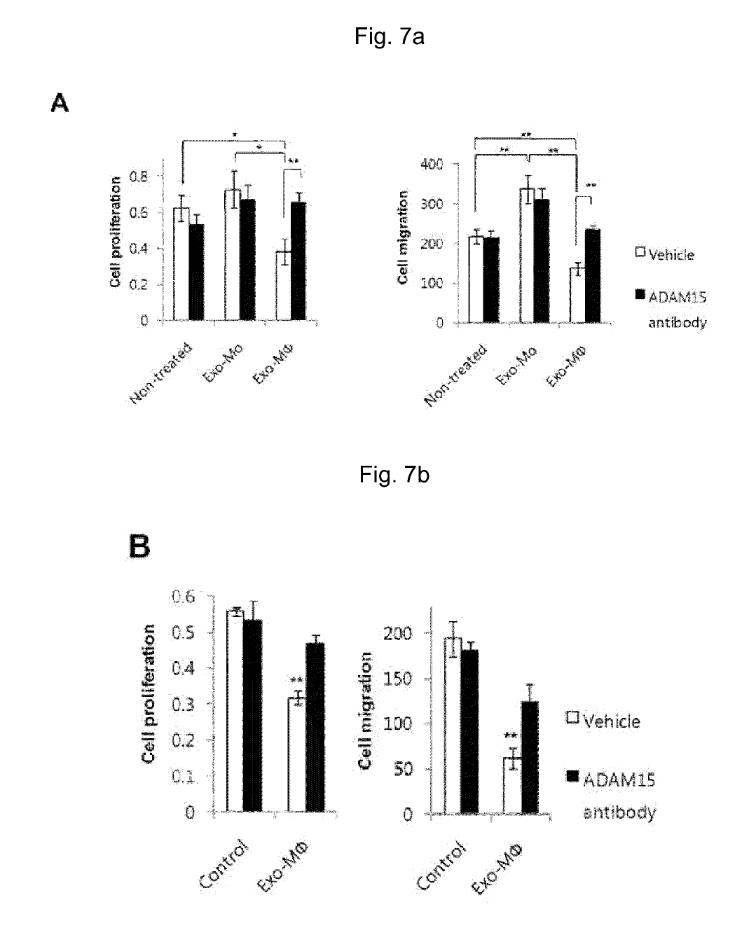

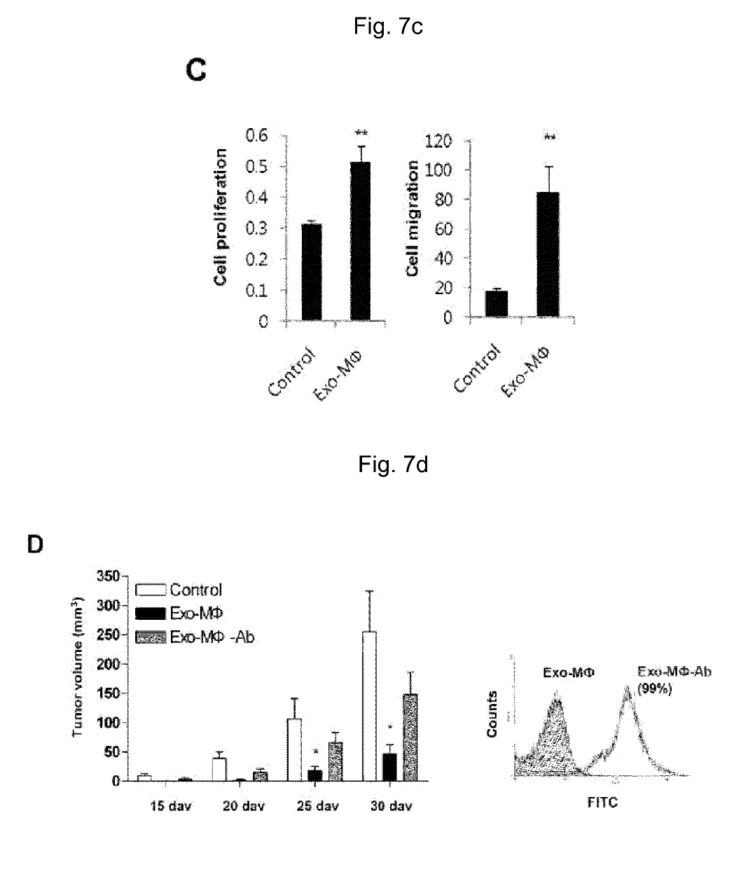

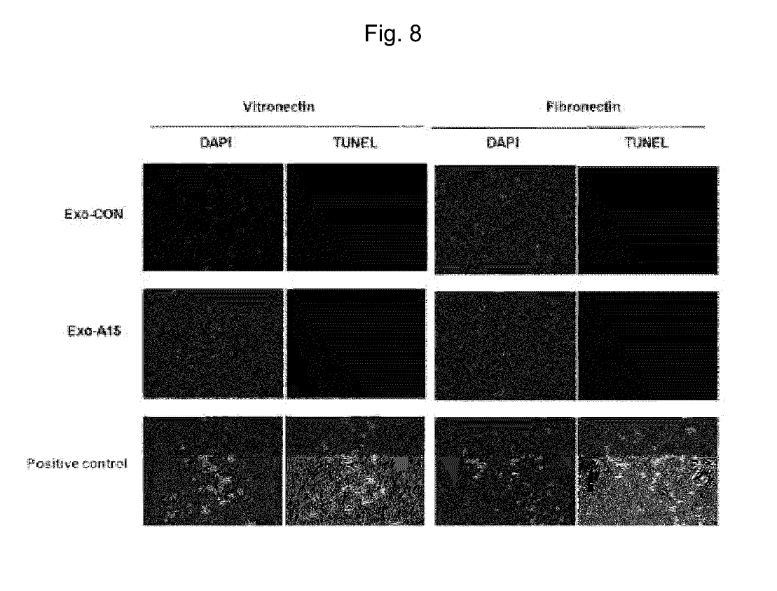

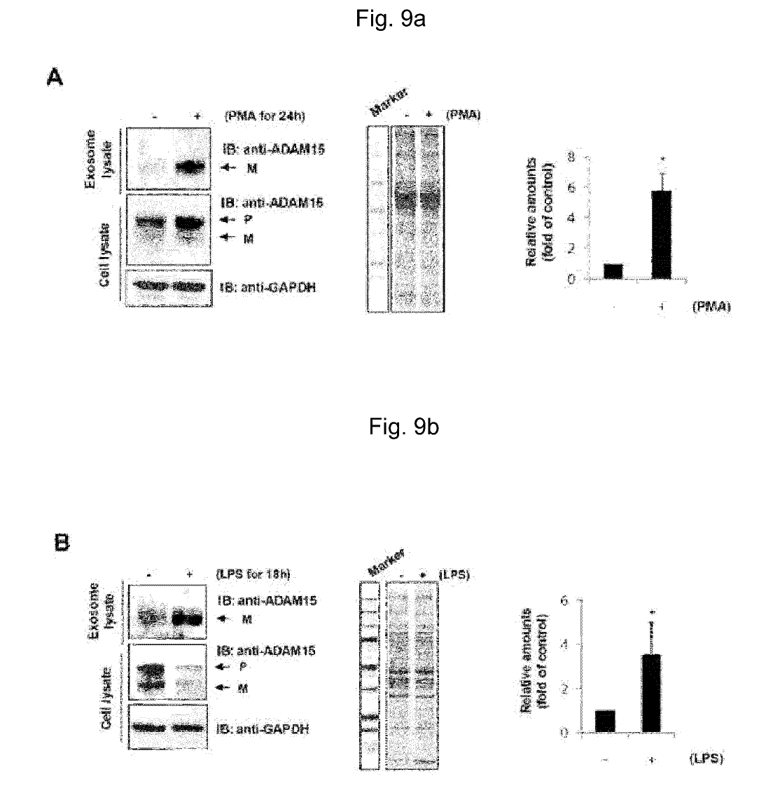

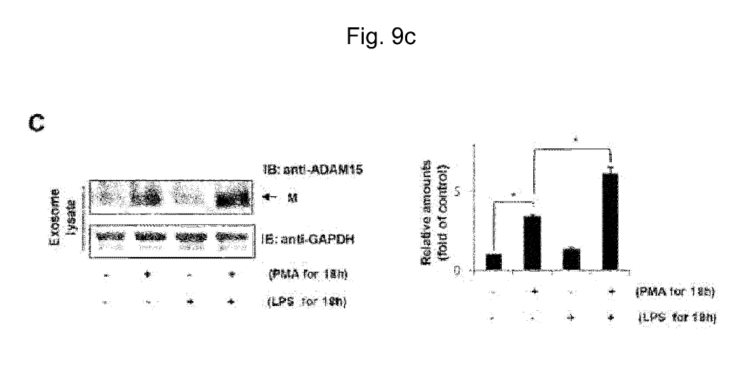

To identify the tumor suppressive role of macrophage-derived exosomal ADAM15, cancer proliferation and migration assays were performed following treatment with exosomes derived from THP-1 monocytes or differentiated macrophages on vitronectin-coated plates. As shown in FIG. 7A, exosomes released from the differentiated macrophages were successful to suppress ovarian cancer MDAH-2774 cell proliferation and migration. These suppressive effects were inhibited by treatment with ADAM15 extracellular domain antibody, indicating the tumor suppressive function of the macrophage-derived exosomal ADAM15. On the other hand, exosomes derived from monocytes slightly stimulated cancer proliferation and migration, and these effects were not affected by treatment with ADAM15 antibody. Obtained results are consistent with previously observed data (FIGS. 5B and 5C). To investigate whether the tumor suppressive function of exosomal ADAM15 released from differentiated macrophages is sustainable without PMA stimulation, macrophage-derived exosomes were purified as described above. It was successful to observe the inhibitory effect of macrophage-derived exosomal ADAM15 on tumor cell proliferation and migration (FIG. 7B). Interestingly, proliferation and migration of macrophage itself were stimulate by the exosomes (FIG. 7C), suggesting an anti-tumor response of human macrophage.

In a nude mouse model, macrophage exosomes also significantly suppressed tumor growth, and the suppressive effects were reduced by preincubating exosomal ADAM15 with ADAM15 antibody prior to injection (FIG. 7D). These data clearly indicate that macrophage-derived exosomal ADAM15 plays a role to inhibit tumor progression in vitro and tumor growth in vivo.

### 3. Discussion

It was attempted in this study to identify ADAM15 release as an exosomal component and to elucidate its biological significance in tumor suppressive mechanism associated with immune function.

Experimental data in this work resulted in several scientific findings. First, ADAM15 is released into extracellular space as an exosomal component, and the release of exosomal ADAM15 is largely induced by PMA-mediated PKC activation in various tumor cells with a corresponding decrease in plasma membrane-associated ADAM15. Second, exosomal ADAM15 binds integrin αvβ3 and inhibits the interaction between integrin αvβ3 and vitronectin. Third, exosomal ADAM15 suppresses vitronectin- and fibronectin-induced tumor proliferation and migration in vitro and tumor growth in vivo. Fourth, the RGD motif-containing a disintegrin-like domain, but not the metalloprotease activity of ADAM15, is the major element for the tumor suppressive function of exosomal ADAM15. Finally, human macrophage is functionally active to release of exosomal ADAM15 and that macrophage-derived exosomal ADAM15 effectively plays tumor suppressive roles.

The present study revealed that tumors release high levels of exosomal ADAM15 with a corresponding decrease in plasma membrane-associated ADAM15 in response to PMA stimulation. Recently, it was reported that exosomal components are differently sorted into exosomes by PMA-mediated PKC activation (Abache et al., 2007). Therefore, ADAM15 might be one of the exosomal components that are sorted into exosomes by PKC activation. PMA-stimulated PKC activation regulates tumor growth and survival (Choi et al., 2006; Feuerstein et al., 1984; Oh et al., 2005; Tahara et al., 2009). Several studies have shown that microvesicles are involved in cellular regulatory mechanisms. For example, exosome-like vesicles containing TNF receptor are secreted from human umbilical vein endothelial cells, and TNF signaling is negatively regulated by the released TNF receptor (Hawari et al., 2004). It has been also suggested that β-catenin-mediated Wnt signaling is inhibited by the release of exosomal β-catenin (Chairoungdua et al., 2010). In this context, it is possible to propose a novel regulatory mechanism for ADAM15-mediated tumorigenesis that is modulated by the release of exosomal ADAM15.

There are several reports demonstrating that the disintegrin-like domain of ADAM15 binds both integrins αvβ3 and α5β1, although the binding is more specific for integrin αvβ3 than for integrin α5β1 depending on cell types (Nath et al., 1999; Zhang et al., 1998). Experimental results in this work revealed that exosomal ADAM15 specifically interacts with integrin αvβ3 in an RGD-dependant manner.

Disintegrins with RGD motifs have been shown to prevent tumor progression through inhibition of integrin-mediated cell-ECM interactions (Chung et al., 2003; Lu et al., 2007; McLane et al., 2004; Trochon-Joseph et al., 2004; Wu et al., 2008). However, it was found that plasma membrane-localized ADAM15 alters neither cell-ECM interactions (FIG. S2A) nor expression of ECM receptors (integrin αvβ3 and α5β1) on the cell surface (FIG. S2B). Instead, released exosomal ADAM15 modulates cell-ECM interaction that is associated with tumor suppressive mechanism. These results led us to postulate that the release of ADAM15 as an exosomal component is a crucial process for ADAM15-mediated tumor suppression.

Experimental data showed that exosomes derived from unstimulated cancer cells are capable of inducing cancer proliferation and migration that are not affected by ADAM15 antibody (FIGS. 5B and 5C), suggesting the presence of other exosomal component(s) than ADAM15. For example, various EGFR ligands are present in exosomes and participate in tumor progression (Higginbotham et al., 2010).

Tumor-suppressive exosomal ADAM15 was found to be released from differentiated human macrophages without PMA stimulation. Differentiation of monocytes into macrophages induces sustainable activation and translocation of PKC (Aihara et al., 1991; Monick et al., 1998). Thus, ADAM15 release may be related to continuous PKC activation in differentiated macrophages. Macrophages play fundamental roles in immune responses against infection or tumors (Adams and Snyderman, 1979; Bock et al., 1991; Braun et al., 1993; Hibbs et al., 1982; Vicetti Miguel et al., 2010; Wu et al., 2009b; Young et al., 1990). Monocyte-derived macrophages are known to be rapidly recruited to the tumor site and classically function to suppress tumor growth (Bock et al., 1991; Braun et al., 1993; Vicetti Miguel et al., 2010; Wu et al., 2009b; Young et al., 1990). Microvesicular functions in immune responses have become increasingly important under both physiological and pathological conditions. Many studies have shown that exosomes derived from dendritic cells or macrophages activate adaptive and innate immunity through the transfer of antigen or MHC-peptide complexes (Lynch et al., 2009; Segura et al., 2007; Taieb et al., 2006; Zeelenberg et al., 2008). Here, we propose a novel anti-tumor immune function of macrophage-derived exosomal ADAM15 that directly suppress tumor progression without triggering other immune cells. It is interesting to note that macrophage-derived exosomes stimulate proliferation and migration of macrophages themselves, in contrast to the results obtained in tumor cells (FIG. 7B). Macrophages can destroy tumorigenic cells through producing tumor cytotoxic factor(s) without affecting non tumorigenic cells (Sone and Fidler, 1981; Sone et al., 1982). A recent study reported that exosomes are effectively internalized by phagocytic cells, while most exosomes are attached to the plasma membrane in non-phagocytic cells (Feng et al., 2010). This work demonstrates a novel tumor suppressive mechanism mediated by exosomal ADAM15 as well as its biological significance as an anti-tumor function of innate immune system.

### 4. References

Abache, T., F. Le Naour, S. Planchon, F. Harper, C. Boucheix, and E. Rubinstein. 2007. The transferrin receptor and the tetraspanin web molecules CD9, CD81, and CD9P-1 are differentially sorted into exosomes after TPA treatment of K562 cells. J Cell Biochem. 102:650-64.
Adams, D. O., and R. Snyderman. 1979. Do macrophages destroy nascent tumors? J Natl Cancer Inst. 62:1341-5.
Aihara, H., Y. Asaoka, K. Yoshida, and Y. Nishizuka. 1991. Sustained activation of protein kinase C is essential to HL-60 cell differentiation to macrophage. Proc Natl Acad Sci USA. 88:11062-6.
Anand, P. K. 2010. Exosomal membrane molecules are potent immune response modulators. Commun Integr Biol. 3:405-8.
Arribas, J., and A. Borroto. 2002. Protein ectodomain shedding. Chem. Rev. 102:4627-38.
Arribas, J., L. Coodly, P. Vollmer, T. K. Kishimoto, S. Rose-John, and J. Massague. 1996. Diverse cell surface protein ectodomains are shed by a system sensitive to metalloprotease inhibitors. J Biol. Chem. 271:11376-82.
Bock, S. N., R. B. Cameron, P. Kragel, J. J. Mule, and S. A. Rosenberg. 1991. Biological and antitumor effects of recombinant human macrophage colony-stimulating factor in mice. Cancer Res. 51:2649-54.
Braun, D. P., M. C. Ahn, J. E. Harris, E. Chu, L. Casey, G. Wilbanks, and K. P. Siziopikou. 1993. Sensitivity of tumoricidal function in macrophages from different anatomical sites of cancer patients to modulation of arachidonic acid metabolism. Cancer Res. 53:3362-8.
Brooks, P. C., R. A. Clark, and D. A. Cheresh. 1994. Requirement of vascular integrin alpha v beta 3 for angiogenesis. Science. 264:569-71.
Brooks, P. C., S. Stromblad, R. Klemke, D. Visscher, F. H. Sarkar, and D. A. Cheresh. 1995. Antiintegrin alpha v beta 3 blocks human breast cancer growth and angiogenesis in human skin. J Clin Invest. 96:1815-22.
Chairoungdua, A., D. L. Smith, P. Pochard, M. Hull, and M. J. Caplan. 2010. Exosome release of beta-catenin: a novel mechanism that antagonizes Wnt signaling. J. Cell Biol. 190:1079-91.
Chen, Q., L. H. Meng, C. H. Zhu, L. P. Lin, H. Lu, and J. Ding. 2008. ADAM15 suppresses cell motility by driving integrin alpha5beta1 cell surface expression via Erk inactivation. Int J Biochem Cell Biol. 40:2164-73.
Choi, S. H., T. Hyman, and P. M. Blumberg. 2006. Differential effect of bryostatin 1 and phorbol 12-myristate 13-acetate on HOP-92 cell proliferation is mediated by down-regulation of protein kinase Cdelta. Cancer Res. 66:7261-9.
Chung, K. H., S. H. Kim, K. Y. Han, Y. D. Sohn, S. I. Chang, K. H. Baek, Y. Jang, D. S. Kim, and I. C. Kang. 2003. Inhibitory effect of salmosin, a Korean snake venom-derived disintegrin, on the integrin alphav-mediated proliferation of SK-MeI-2 human melanoma cells. J Pharm Pharmacol. 55:1577-82.
Cocucci, E., G. Racchetti, and J. Meldolesi. 2009. Shedding microvesicles: artefacts no more. Trends Cell Biol. 19:43-51.
Daigneault, M., J. A. Preston, H. M. Marriott, M. K. Whyte, and D. H. Dockrell. 2010. The identification of markers of macrophage differentiation in PMA-stimulated THP-1 cells and monocyte-derived macrophages. PLoS One. 5:e8668.
Feng, D., W. L. Zhao, Y. Y. Ye, X. C. Bai, R. Q. Liu, L. F. Chang, Q. Zhou, and S. F. Sui. 2010. Cellular internalization of exosomes occurs through phagocytosis. Traffic. 11:675-87.
Feuerstein, N., A. Sahai, W. B. Anderson, D. S. Salomon, and H. L. Cooper. 1984. Differential phosphorylation events associated with phorbol ester effects on acceleration versus inhibition of cell growth. Cancer Res. 44:5227-33.
Gutwein, P., A. Stoeck, S. Riedle, D. Gast, S. Runz, T. P. Condon, A. Marme, M. C. Phong, O. Linderkamp, A. Skorokhod, and P. Altevogt. 2005. Cleavage of L1 in exosomes and apoptotic membrane vesicles released from ovarian carcinoma cells. Clin Cancer Res. 11:2492-501.
Hahn, D., A. Pischitzis, S. Roesmann, M. K. Hansen, B. Leuenberger, U. Luginbuehl, and E. E. Sterchi. 2003. Phorbol 12-myristate 13-acetate-induced ectodomain shedding and phosphorylation of the human meprinbeta metalloprotease. J Biol. Chem. 278:42829-39.
Hawari, F. I., F. N. Rouhani, X. Cui, Z. X. Yu, C. Buckley, M. Kaler, and S. J. Levine. 2004. Release of full-length 55-kDa TNF receptor 1 in exosome-like vesicles: a mechanism for generation of soluble cytokine receptors. Proc Natl Acad Sci USA. 101:1297-302.
Herren, B., E. W. Raines, and R. Ross. 1997. Expression of a disintegrin-like protein in cultured human vascular cells and in vivo. FASEB J. 11:173-80.
Hibbs, J. B., Jr., D. L. Granger, J. L. Cook, and A. M. Lewis, Jr. 1982. Activated macrophage mediated cytotoxicity for transformed target cells. Adv Exp Med. Biol. 146:315-35.
Higginbotham, J. N., M. Demory Beckler, J. D. Gephart, J. L. Franklin, G. Bogatcheva, G. J. Kremers, D. W. Piston, G. D. Ayers, R. E. McConnell, M. J. Tyska, and R. J. Coffey. 2010. Amphiregulin exosomes increase cancer cell invasion. Curr Biol. 21:779-86.
Kratzschmar, J., L. Lum, and C. P. Blobel. 1996. Metargidin, a membrane-anchored metalloprotease-disintegrin protein with an RGD integrin binding sequence. J Biol. Chem. 271:4593-6.
Landen, C. N., T. J. Kim, Y. G. Lin, W. M. Merritt, A. A. Kamat, L. Y. Han, W. A. Spannuth, A. M. Nick, N. B. Jennnings, M. S. Kinch, D. Tice, and A. K. Sood. 2008. Tumor-selective response to antibody-mediated targeting of alphavbeta3 integrin in ovarian cancer. Neoplasia. 10:1259-67.
Linden, D. J., and J. A. Connor. 1991. Participation of postsynaptic PKC in cerebellar long-term depression in culture. Science. 254:1656-9.
Lu, D., S. Xie, M. B. Sukkar, X. Lu, M. F. Scully, and K. F. Chung. 2007. Inhibition of airway smooth muscle adhesion and migration by the disintegrin domain of ADAM-15. Am J Respir Cell Mol. Biol. 37:494-500.
Lynch, S., S. G. Santos, E. C. Campbell, A. M. Nimmo, C. Botting, A. Prescott, A. N. Antoniou, and S. J. Powis. 2009. Novel MHC class I structures on exosomes. J. Immunol. 183:1884-91.
McLane, M. A., E. E. Sanchez, A. Wong, C. Paquette-Straub, and J. C. Perez. 2004. Disintegrins. Curr Drug Targets Cardiovasc Haematol Disord. 4:327-55.
Mochizuki, S., and Y. Okada. 2007. ADAMs in cancer cell proliferation and progression. Cancer Sci. 98:621-8.
Monick, M. M., A. B. Carter, G. Gudmundsson, L. J. Geist, and G. W. Hunninghake. 1998. Changes in PKC isoforms in human alveolar macrophages compared with blood monocytes. Am J. Physiol. 275:L389-97.
Najy, A. J., K. C. Day, and M. L. Day. 2008a. ADAM15 supports prostate cancer metastasis by modulating tumor cell-endothelial cell interaction. Cancer Res. 68:1092-9.
Najy, A. J., K. C. Day, and M. L. Day. 2008b. The ectodomain shedding of E-cadherin by ADAM15 supports ErbB receptor activation. J Biol. Chem. 283:18393-401.
Nath, D., P. M. Slocombe, P. E. Stephens, A. Warn, G. R. Hutchinson, K. M. Yamada, A. J. Docherty, and G. Murphy. 1999. Interaction of metargidin (ADAM-15) with alphavbeta3 and alpha5beta1 integrins on different haemopoietic cells. J Cell Sci. 112 (Pt 4):579-87.
Oh, J., Y. S. Choi, J. W. Kim, J. Y. Park, S. W. Kim, K. K. Park, and Y. K. Pak. 2005. Inhibition of low density lipoprotein receptor expression by long-term exposure to phorbol ester via p38 mitogen-activated protein kinase pathway. J Cell Biochem. 96:786-94.
Schafer, B., A. Gschwind, and A. Ullrich. 2004a. Multiple G-protein-coupled receptor signals converge on the epidermal growth factor receptor to promote migration and invasion. Oncogene. 23:991-9.
Schafer, B., B. Marg, A. Gschwind, and A. Ullrich. 2004b. Distinct ADAM metalloproteinases regulate G protein-coupled receptor-induced cell proliferation and survival. J Biol. Chem. 279:47929-38.
Segura, E., C. Guerin, N. Hogg, S. Amigorena, and C. Thery. 2007. CD8+ dendritic cells use LFA-1 to capture MHC-peptide complexes from exosomes in vivo. J. Immunol. 179:1489-96.
Sone, S., and I. J. Fidler. 1981. In vitro activation of tumoricidal properties in rat alveolar macrophages by synthetic muramyl dipeptide encapsulated in liposomes. Cell Immunol. 57:42-50.
Sone, S., S. Moriguchi, E. Shimizu, F. Ogushi, and E. Tsubura. 1982. In vitro generation of tumoricidal properties in human alveolar macrophages following interaction with endotoxin. Cancer Res. 42:2227-31.
Tahara, E., H. Kadara, L. Lacroix, D. Lotan, and R. Lotan. 2009. Activation of protein kinase C by phorbol 12-myristate 13-acetate suppresses the growth of lung cancer cells through KLF6 induction. Cancer Biol Ther. 8:801-7.
Taieb, J., N. Chaput, N. Schartz, S. Roux, S, Novault, C. Menard, F. Ghiringhelli, M. Terme, A. F. Carpentier, G. Darrasse-Jeze, F. Lemonnier, and L. Zitvogel. 2006. Chemoimmunotherapy of tumors: cyclophosphamide synergizes with exosome based vaccines. J. Immunol. 176:2722-9.
Thery, C., M. Boussac, P. Veron, P. Ricciardi-Castagnoli, G. Raposo, J. Garin, and S. Amigorena. 2001. Proteomic analysis of dendritic cell-derived exosomes: a secreted subcellular compartment distinct from apoptotic vesicles. J. Immunol. 166:7309-18.
Thery, C., M. Ostrowski, and E. Segura. 2009. Membrane vesicles as conveyors of immune responses. Nat Rev Immunol. 9:581-93.
Toquet, C., A. Colson, A. Jarry, S. Bezieau, C. Volteau, P. Boisseau, D. Merlin, C. L. Laboisse, and J. F. Mosnier. 2010. ADAM15 to alpha5beta1 integrin switch in colon carcinoma cells: A late event in cancer progression associated with tumor dedifferentiation and poor prognosis. Int J. Cancer.
Trochon-Joseph, V., D. Martel-Renoir, L. M. Mir, A. Thomaidis, P. Opolon, E. Connault, H. Li, C. Grenet, F. Fauvel-Lafeve, J. Soria, C. Legrand, C. Soria, M. Perricaudet, and H. Lu. 2004. Evidence of antiangiogenic and antimetastatic activities of the recombinant disintegrin domain of metargidin. Cancer Res. 64:2062-9.
Ungerer, C., K. Doberstein, C. Burger, K. Hardt, W. H. Boehncke, B. Bohm, J. Pfeilschifter, R. Dummer, D. Mihic-Probst, and P. Gutwein. 2010. ADAM15 expression is downregulated in melanoma metastasis compared to primary melanoma. Biochem Biophys Res Commun. 401:363-9.
Valadi, H., K. Ekstrom, A. Bossios, M. Sjostrand, J. J. Lee, and J. O. Lotvall. 2007. Exosome-mediated transfer of mRNAs and microRNAs is a novel mechanism of genetic exchange between cells. Nat Cell Biol. 9:654-9.
van Kilsdonk, J. W., L. C. van Kempen, G. N. van Muijen, D. J. Ruiter, and G. W. Swart. 2010. Soluble adhesion molecules in human cancers: sources and fates. Eur J. Cell Biol. 89:415-27.
Vecchi, M., J. Baulida, and G. Carpenter. 1996. Selective cleavage of the heregulin receptor ErbB-4 by protein kinase C activation. J Biol. Chem. 271:18989-95.
Vicetti Miguel, R. D., T. L. Cherpes, L. J. Watson, and K. C. McKenna. 2010. CTL induction of tumoricidal nitric oxide production by intratumoral macrophages is critical for tumor elimination. J. Immunol. 185:6706-18.
Whatling, C., H. Bjork, S. Gredmark, A. Hamsten, and P. Eriksson. 2004. Effect of macrophage differentiation and exposure to mildly oxidized LDL on the proteolytic repertoire of THP-1 monocytes. J Lipid Res. 45:1768-76.
Wolfsberg, T. G., P. Primakoff, D. G. Myles, and J. M. White. 1995. ADAM, a novel family of membrane proteins containing A Disintegrin And Metalloprotease domain: multipotential functions in cell-cell and cell-matrix interactions. J. Cell Biol. 131:275-8.
Wu, J., M. C. Wu, L. F. Zhang, J. Y. Lei, L. Feng, and J. Jin. 2009a. Identification of binding peptides of the ADAM15 disintegrin domain using phage display. J. Biosci. 34:213-20.
Wu, J., L. Zhang, X. Ma, X. Zhang, and J. Jin. 2008. Screening cellular proteins involved in the anti-proliferative effect of the ADAM15 disintegrin domain in murine melanoma cells. Oncol Rep. 20:669-75.
Wu, Q. L., I. N. Buhtoiarov, P. M. Sondel, A. L. Rakhmilevich, and E. A. Ranheim. 2009b. Tumoricidal effects of activated macrophages in a mouse model of chronic lymphocytic leukemia. J. Immunol. 182:6771-8.
Young, D. A., L. D. Lowe, and S. C. Clark. 1990. Comparison of the effects of IL-3, granulocyte-macrophage colony-stimulating factor, and macrophage colony-stimulating factor in supporting monocyte differentiation in culture. Analysis of macrophage antibody-dependent cellular cytotoxicity. J. Immunol. 145:607-15.
Zeelenberg, I. S., M. Ostrowski, S. Krumeich, A. Bobrie, C. Jancic, A. Boissonnas, A. Delcayre, J. B. Le Pecq, B. Combadiere, S. Amigorena, and C. Thery. 2008. Targeting tumor antigens to secreted membrane vesicles in vivo induces efficient antitumor immune responses. Cancer Res. 68:1228-35.
Zhang, X. P., T. Kamata, K. Yokoyama, W. Puzon-McLaughlin, and Y. Takada. 1998. Specific interaction of the recombinant disintegrin-like domain of MDC-15 (metargidin, ADAM-15) with integrin alphavbeta3. J Biol. Chem. 273:7345-50.
Zibert, J. R., K. Wallbrecht, M. Schon, L. M. Mir, G. K. Jacobsen, V. Trochon-Joseph, C. Bouquet, L. S. Villadsen, R. Cadossi, L. Skov, and M. P. Schon. 2011. Halting angiogenesis by non-viral somatic gene therapy alleviates psoriasis and murine psoriasiform skin lesions. J Clin Invest. 121:410-21.

Although the present disclosure has been described in detail with reference to the specific features, it will be apparent to those skilled in the art that this description is only for examples of the present disclosure and does not limit the scope of the present disclosure. These embodiments are only for illustrating the present disclosure more specifically, and it is apparent to those skilled in the art that the scope of the present disclosure is not limited by these embodiments.

1. A method for preparing microvesicular ADAM15 from mammalian cells, the method comprising:
(a) activating protein kinase C (PKC) of isolated mammalian cells; and
(b) isolating exosomes containing ADAM15 from the mammalian cells.

2. The method of claim 1, wherein step (a) is conducted by using phorbol-12-myristate-13-acetate (PMA), 1-oleoyl-2-acetylgylcerol, 1-stearoyl-2-arachidonoyl-sn-glycerol, 1,2-didecanoylglycerol, 1,2-dioctanoyl-sn-glycerol, (2S,5S)-8-decylbenzolactam V, 6-(N-decylamino)-4-hydroxymethylindole, 7-octylindolactam V, 1,2-di-O-octanoyl-3-O-β-D-galactopyranosyl-rac-glycerol, 12-deoxyphorbol 13-phenylacetate, 1-oreoyl-2-acetyl-sn-glycerol (OAG), 1-oreoyl-2-O-acetyl-3-β-D-galactopyranocyl sn glycerol, 1-stearoyl-2-linoleoyl-sn-glycerol, 8-hydroxy-[S-(E,Z,Z,Z)]-5,9,11,14-eicosatetraenoic acid, arachidonic acid, cholesterol 3-sulfate, oleic acid, phorbol 12,13-diacetate (PDA), phorbol 12,13-dibutyrate (PDBu), phorbol 12,13-didecanoate (PDD), 4α-phorbol-12,13-didecanoate, L-α-phosphatidylinositol-3,4-bisphosphate, L-α-phosphatidylinositol-4,5-bisphosphate, L-α-phosphatidyl inositol-3,4,5-triphosphate, 1-stearoyl-2-arachidonoyl-sn-glycerol, 5-chloro-N-heptyl-naphtalene-1-sulfonamide, resiniferatoxin (RTX), resiniferonol 9,13,14-ortho-phenylacetate (ROPA), 12-deoxyphorbol 13-phenylacate 20-acetate, indolactam V, 20-dibenzoate, phorbol 12,13-dihexanoate, phorbol-12,13-didecanoate, 1-hexylindolactam-V10, 6,11,12,14-tetrahydroxy-abieta-5,8,11,13-tetraene-7-one (coleon U), 8-octyl-benzolactam-V9, acetyl-L-carnitine, chloroform, retinoic acid, or phorbol ester 12-O-tetradecanoyl-phorbol-13-acetate.

3. The method of claim 1, wherein the mammalian cells are human cells.

4. The method of claim 1, wherein the mammalian cells are normal cells or tumor cells.

5. The method of claim 1, wherein the mammalian cells are immune cells.

6. The method of claim 5, wherein the immune cells are macrophages.

7. A method for inducing microvesicular ADAM15 generation, the method comprising a PKC activator.

8. The method of claim 7, wherein the PKC activator is phorbol-12-myristate-13-acetate (PMA), 1-oleoyl-2-acetylgylcerol, 1-stearoyl-2-arachidonoyl-sn-glycerol, 1,2-didecanoylglycerol, 1,2-dioctanoyl-sn-glycerol, (2S,5S)-8-decylbenzolactam V, 6-(N-decylamino)-4-hydroxymethylindole, 7-octylindolactam V, 1,2-di-O-octanoyl-3-O-β-D-galactopyranosyl-rac-glycerol, 12-deoxyphorbol 13-phenylacetate, 1-oreoyl-2-acetyl-sn-glycerol (OAG), 1-oreoyl-2-O-acetyl-3-β-D-galactopyranocyl sn glycerol, 1-stearoyl-2-linoleoyl-sn-glycerol, 8-hydroxy-[S-(E,Z,Z,Z)]-5,9,11,14-eicosatetraenoic acid, arachidonic acid, cholesterol 3-sulfate, oleic acid, phorbol 12,13-diacetate (PDA), phorbol 12,13-dibutyrate (PDBu), phorbol 12,13-didecanoate (PDD), 4α-phorbol-12,13-didecanoate, L-α-phosphatidylinositol-3,4-bisphosphate, L-α-phosphatidylinositol-4,5-bisphosphate, L-α-phosphatidyl inositol-3,4,5-triphosphate, 1-stearoyl-2-arachidonoyl-sn-glycerol, 5-chloro-N-heptyl-naphtalene-1-sulfonamide, resiniferatoxin (RTX), resiniferonol 9,13,14-ortho-phenylacetate (ROPA), 12-deoxyphorbol 13-phenylacate 20-acetate, indolactam V, 20-dibenzoate, phorbol 12.13,13-di hexanoate, phorbol-12,13-didecanoate, 1-hexylindolactam-V10, 6,11,12,14-tetrahydroxy-abieta-5,8,11,13-tetraene-7-one (coleon U), 8-octyl-benzolactam-V9, acetyl-L-carnitine, chloroform, retinoic acid, or phorbol ester 12-O-tetradecanoyl-phorbol-13-acetate.

9. A method for preparing microvesicular ADAM15, the method comprising:
(a) incubating macrophages to generate exosomes containing ADAM15; and
(b) isolating the exosomes containing ADAM15.

10. The method of claim 9, further comprising, prior to step (a), differentiating monocytes into the macrophages

11. The method of claim 10, wherein the differentiating is conducted by using a PKC activator.

12. The method of claim 9, wherein the macrophages are cells treated with an immune adjuvant.

13. The method of claim 12, wherein the immune adjuvant is liposome, lipopolysaccharide (LPS), double-strand RNA, single-strand DNA, or unmethylated CpG dinucleotide.

14. A Method for inducing microvesicular ADAM15 generation from macrophages, the method comprising an immune adjuvant.

15. The Method of claim 14, wherein the immune adjuvant is liposome, lipopolysaccharide (LPS), or immunostimulatory oligonucleotide.


SEQUENCE LISTING (2 sequences)

SEQ ID NO: 1 - DNA, 21 bp, Artificial Sequence
  Synthetic Construct
atgcggctggcgctgctctgg

SEQ ID NO: 2 - DNA, 21 bp, Artificial Sequence
  Synthetic Construct
tcagaggtagagcgaggacac